SEQUENCE LISTING (28 sequences)

SEQ ID NO: 1 - protein, 5 aa, synthetic construct
DYGMH

SEQ ID NO: 2 - protein, 17 aa, synthetic construct
YISSGSYTIYSADSVKG

SEQ ID NO: 3 - protein, 13 aa, synthetic construct
RAPNSFYEYYFDY

SEQ ID NO: 4 - protein, 16 aa, synthetic construct
SASSKHTNLYWSRHMY

SEQ ID NO: 5 - protein, 7 aa, synthetic construct
LTSNRAT

SEQ ID NO: 6 - protein, 9 aa, synthetic construct
QQWSSNPFT

SEQ ID NO: 7 - protein, 122 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 8 - protein, 452 aa, synthetic construct
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 112 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 10 - protein, 219 aa, synthetic construct
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SYAMH

SEQ ID NO: 12 - protein, 17 aa, synthetic construct
VISYAGEVKYYADSVKG

SEQ ID NO: 13 - protein, 12 aa, synthetic construct
DPLPLHYYGMDV

SEQ ID NO: 14 - protein, 13 aa, synthetic construct
SGSSSNIGRRPVN

SEQ ID NO: 15 - protein, 7 aa, synthetic construct
SQNQRPS

SEQ ID NO: 16 - protein, 11 aa, synthetic construct
AVWDDIGRVLQ

SEQ ID NO: 17 - protein, 121 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 18 - protein, 451 aa, synthetic construct
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 110 aa, synthetic construct
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVL

SEQ ID NO: 20 - protein, 216 aa, synthetic construct
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 21 - DNA, 366 bp, synthetic construct
  source 1..366: mol_type = other DNA
gaggtgcagctggtggaatccggcggaggactggtgcagcctggcggctccctgagactgtcttgcgccgcctccggcttcacattctccgactacggcatgcactgggtccgacaggcccctggaaagggcctggaatgggtggcctacatctcctccggctcctacaccatctactccgccgactccgtgaagggccggttcaccatctcccgggacaacgccaagaactccctgtacctgcagatgaactccctgcgggccgaggacacagccgtgtactactgtgccagacgggcccctaactccttctacgagtactacttcgactactggggccagggcaccaccgtgaccgtgtcctct

SEQ ID NO: 22 - DNA, 1359 bp, synthetic construct
  source 1..1359: mol_type = other DNA
gaggtgcagctggtggaatccggcggaggactggtgcagcctggcggctccctgagactgtcttgcgccgcctccggcttcacattctccgactacggcatgcactgggtccgacaggcccctggaaagggcctggaatgggtggcctacatctcctccggctcctacaccatctactccgccgactccgtgaagggccggttcaccatctcccgggacaacgccaagaactccctgtacctgcagatgaactccctgcgggccgaggacacagccgtgtactactgtgccagacgggcccctaactccttctacgagtactacttcgactactggggccagggcaccaccgtgaccgtgtcctctgctagcaccaaaggtccgagcgtttttccgctggcaccgagcagcaaaagcacctctggtggcaccgcagcactgggttgtctggtgaaagattattttccggaaccggttaccgtttcttggaatagcggtgcactgaccagcggtgttcatacctttccggcagttctgcagagcagcggtctgtatagcctgtctagcgttgttaccgttccgagcagcagcctgggcacccagacctatatttgcaatgtgaatcataaaccgagcaatacaaaagttgataaacgcgttgaaccgaaaagctgtgacaaaactcacacgtgcccaccgtgcccagcacctgagttcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccagcatcgagaaaaccatctccaaagccaaagggcagccccgagaaccacaggtctgcaccctgcccccatcccgggaggagatgaccaagaaccaggtcagcctgagctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 23 - DNA, 336 bp, synthetic construct
  source 1..336: mol_type = other DNA
cagatcgtgctgacccagtcccctgccaccctgtccctgagccctggcgagagagccaccctgagctgctccgcctcctccaagcacaccaacctgtactggtcccggcacatgtactggtatcagcagaagcccggccaggcccctcggctgctgatctacctgacctctaaccgggccaccggcatccctgccagattctccggctctggctccggcaccgacttcaccctgaccatctccagcctggaacccgaggacttcgccgtgtactactgccagcagtggtcctccaaccccttcaccttcggccagggcaccaagctggaaatcaag

SEQ ID NO: 24 - DNA, 660 bp, synthetic construct
  source 1..660: mol_type = other DNA
cagatcgtgctgacccagtcccctgccaccctgtccctgagccctggcgagagagccaccctgagctgctccgcctcctccaagcacaccaacctgtactggtcccggcacatgtactggtatcagcagaagcccggccaggcccctcggctgctgatctacctgacctctaaccgggccaccggcatccctgccagattctccggctctggctccggcaccgacttcaccctgaccatctccagcctggaacccgaggacttcgccgtgtactactgccagcagtggtcctccaaccccttcaccttcggccagggcaccaagctggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 25 - DNA, 363 bp, synthetic construct
  source 1..363: mol_type = other DNA
caggtgcagctggtggagtctggaggaggcgtggtccagcctgggaggtccctgagactctcctgtgcagcctctggattcaccttcagtagctatgctatgcactgggtccgccaggctccaggcaaggggctggagtgggtggcagttatatcatatgcgggggaggtgaaatactacgcagactccgtgaagggccgattcaccatctccagagacaattccaagaacacgctgtatctgcaaatgaacagcctgagagctgaggacacggctgtgtattactgtgcgagagacccgctaccgctacattactacggtatggacgtctggggccaagggaccacggtcaccgtctcctca

SEQ ID NO: 26 - DNA, 1356 bp, synthetic construct
  source 1..1356: mol_type = other DNA
caggtgcagctggtggagtctggaggaggcgtggtccagcctgggaggtccctgagactctcctgtgcagcctctggattcaccttcagtagctatgctatgcactgggtccgccaggctccaggcaaggggctggagtgggtggcagttatatcatatgcgggggaggtgaaatactacgcagactccgtgaagggccgattcaccatctccagagacaattccaagaacacgctgtatctgcaaatgaacagcctgagagctgaggacacggctgtgtattactgtgcgagagacccgctaccgctacattactacggtatggacgtctggggccaagggaccacggtcaccgtctcctcagcgtcgaccaaaggtccgagcgtgtgcccgctggcaccgagcagcaaaagcacctctggtggcaccgcagcactgggttgtctggtgaaagattattttccggaaccggttaccgtttcttggaatagcggtgcactgaccagcggtgttcatacctttccggcagtcctgcagagcagcggtctgtatagcctgtctagcgttgttaccgttccgagcagcagcctgggcacccagacctatatttgcaatgtgaatcataaaccgagcaataccaaagttgataaacgcgttgaaccgaaaagcgtggacaaaactcacacgtgcccaccgtgcccagcacctgagttcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccagcatcgagaaaaccatctccaaagccaaagggcagccccgagaaccacaggtctacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaatga

SEQ ID NO: 27 - DNA, 330 bp, synthetic construct
  source 1..330: mol_type = other DNA
cagtctgtgctgactcagcctccctcagcgtctgggacccccgggcagagggtcaccatctcttgttctggaagcagctccaatatcggaaggaggcctgtaaactggtaccagcagctcccaggaacggcccccaaactcctcatctatagtcagaatcagcggccctcaggggtccctgaccgattctctggctcccagtctggcacctcagcctccctggccatcagtgggctccagtctgaggatgaggctgattatttctgtgcagtttgggatgacatcggccgcgtcctgcagttaggcggaggcacccagctgaccgtccta

SEQ ID NO: 28 - DNA, 651 bp, synthetic construct
  source 1..651: mol_type = other DNA
cagtctgtgctgactcagcctccctcagcgtctgggacccccgggcagagggtcaccatctcttgttctggaagcagctccaatatcggaaggaggcctgtaaactggtaccagcagctcccaggaacggcccccaaactcctcatctatagtcagaatcagcggccctcaggggtccctgaccgattctctggctcccagtctggcacctcagcctccctggccatcagtgggctccagtctgaggatgaggctgattatttctgtgcagtttgggatgacatcggccgcgtcctgcagttaggcggaggcacccagctgaccgtcctaggtcagcccaaggcggccccctcggtcactctgttcccgccctgctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaagtgtcatga

(19) United States
(12) Patent Application Publication
(10) Pub. No.: US 2024/0392005 A1
(43) Pub. Date: Nov. 28, 2024

(54) PD-1/TIGIT BINDING PROTEINS FOR CANCER TREATMENT

(71) Applicant: MedImmune, LLC, Gaithersburg, MD (US)

(72) Inventors: Moritz DRACHSLER, Cambridge (GB); KyoungSoo LIM, Wilmington, DE (US); Chang GONG, Wilmington, DE (US)

(21) Appl. No.: 18/633,844

(22) Filed: Apr. 12, 2024

Related U.S. Application Data
(60) Provisional application No. 63/495,837, filed on Apr. 13, 2023.
(60) Provisional application No. 63/592,164, filed on Oct. 21, 2023.

(51) Int. Cl. C07K 16/28; A61P 35/00; C07K 16/18

(52) U.S. Cl. CPC C07K 16/2818; A61P 35/00; C07K 16/18; C07K 2317/24; C07K 2317/31; C07K 2317/52

(57) ABSTRACT

The disclosure relates to methods of treating cancer by administering binding proteins, including antibodies, that bind to Programmed Death-1 (“PD-1”) and T cell immunoreceptor with Ig and ITIM domains (“TIGIT”) to a subject in an amount from about 70 mg to about 1500 mg.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 9, 2024, is named “0098-0093 SL.xml” and is 32,441 bytes in size. The sequence listing contained in this .XML file is part of the specification and is hereby incorporated by reference herein in its entirety.

### FIELD

The present disclosure provides methods for treating cancer in a subject, comprising administering to the subject a bispecific binding protein that specifically binds to Programmed Death-1 (PD-1) and T cell immunoreceptor with Ig and ITIM domains (TIGIT) in an amount from about 70 mg to about 1500 mg. The present disclosure further provides methods of making such bispecific binding protein, and compositions, including pharmaceutical compositions, comprising such bispecific protein.

### BACKGROUND

Programmed Death-1 (PD-1) is an approximately 31 kD type I membrane protein that is a member of the extended CD28/CTLA4 family of T cell regulators (see Ishida et al., Induced Expression of PD-1, A Novel Member of the Immunoglobulin Gene Superfamily, Upon Programmed Cell Death, EMBO J. 1992, 11: 3887-95). PD-1 is expressed on activated T cells, B cells, and monocytes and at low levels in natural killer (NK) T cells. PD-1 is a well-validated target for immune mediated therapy in oncology. Antagonistic inhibition of the PD-1/PD-L1 interaction increases T cell activation, enhancing recognition and elimination of tumour cells by the host immune system.

T cell immunoreceptor with Ig and ITIM domains (TIGIT) is an immune receptor present on some T cells and Natural Killer Cells (NK). TIGIT is upregulated by immune cells, including activated T cells, natural killer cells, and regulatory T cells.

### SUMMARY

The present disclosure provides a method for treating a cancer in a subject comprising administering to the subject a bispecific binding proteins that specifically binds to PD-1 and TIGIT in an amount from about 70 mg to about 1500 mg. The bispecific binding protein comprises: a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

In some aspects, the amount of bispecific binding protein administered is about 70 mg, about 150 mg, about 210 mg, about 450 mg, about 750 mg, about 800 mg, about 850 mg, about 900 mg, about 950 mg, about 1000 mg, about 1250 mg, or about 1500 mg. In some aspects, the amount of bispecific binding protein administered is about 750 mg. In some aspects, the amount of bispecific binding protein administered is about 1500 mg.

In some aspects, the bispecific binding protein is administered once per treatment cycle. In some aspects, the treatment cycle is about 7 days, about 14 days, about 21 days, about 28 days, or about 35 days. In some aspects, the treatment cycle is about 7 days. In some aspects, the treatment cycle is repeated for up to 35 cycles.

In some aspects, the bispecific binding protein is administered to the subject as a monotherapy. In some aspects, the bispecific binding protein is administered by an intravenous infusion (IV). In some aspects, the subject has not received a prior line of systemic therapy. In some aspects, the subject has previously received a chemotherapy.

In some aspects, the cancer comprises a cancer cell which expresses PD-L1.

In some aspects, the first binding domain of the bispecific binding protein that specifically binds to PD-1 comprises a heavy chain variable domain having the amino acid sequence of SEQ ID NO:7 and a light chain variable domain having the amino acid sequence of SEQ ID NO:9.

In some aspects, the first binding domain of the bispecific binding protein that specifically binds to PD-1 comprises a heavy chain variable domain having an amino acid sequence that is at least 90% identical to the amino acid sequence of SEQ ID NO:7 and a light chain variable domain having an amino acid sequence that is at least 90% identical to the amino acid sequence of SEQ ID NO:9.

In some aspects, the first binding domain of the bispecific binding protein that specifically binds to PD-1 comprises a heavy chain having the amino acid sequence of SEQ ID NO:8 and a light chain having the amino acid sequence of SEQ ID NO:10.

In some aspects, the first binding domain of the bispecific binding protein that specifically binds to PD-1 comprises a heavy chain having an amino acid sequence that is at least 90% identical to the amino acid sequence of SEQ ID NO:8 and a light chain having an amino acid sequence that is at least 90% identical to the amino acid sequence of SEQ ID NO:10.

In some aspects, the second binding domain of the bispecific binding protein that specifically binds to TIGIT comprises a heavy chain variable domain having the amino acid sequence of SEQ ID NO:17 and a light chain variable domain having the amino acid sequence of SEQ ID NO:19.

In some aspects, the second binding domain of the bispecific binding protein that specifically binds to TIGIT comprises a heavy chain variable domain having an amino acid sequence that is at least 90% identical to the amino acid sequence of SEQ ID NO:17 and a light chain variable domain having an amino acid sequence that is at least 90% identical to the amino acid sequence of SEQ ID NO:19.

In some aspects, the second binding domain of the bispecific binding protein that specifically binds to TIGIT comprises a heavy chain having the amino sequence of SEQ ID NO:18 and a light chain having the amino acid sequence of SEQ ID NO:20.

In some aspects, the second binding domain of the bispecific binding protein that specifically binds to TIGIT comprises a heavy chain having an amino acid sequence that is at least 90% identical to the amino acid sequence of SEQ ID NO:18 and a light chain having an amino acid sequence that is at least 90% identical to the amino acid sequence of SEQ ID NO:20.

In some aspects, the bispecific binding protein is a human or humanized bispecific antibody or antigen-binding fragment thereof.

In some aspects, the bispecific binding protein comprises a variant Fc region. In some aspects, the variant Fc region of the bispecific binding protein comprises at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 262I, 262A, 262T, 262E, 263I, 263A, 263T, 263M, 264L, 264I, 264W, 264T, 264R, 264F, 264M, 264Y, 264E, 265A, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

In some aspects, the variant Fc region of the bispecific binding protein comprises one or more amino acid substitutions at positions selected from 428 and 434 as numbered by the EU index as set forth in Kabat. In some aspects, the variant Fc region of the bispecific binding protein comprises one or more amino acid substitutions selected from 428L, 428F, 434A, 424F, 434W, and 434Y.

In some aspects, the variant Fc region of the bispecific binding protein comprises a YTE mutation (M252Y/S254T/T256E). In some aspects, the Fc variant region of the bispecific binding protein comprises a L234F/L235E/P331S triple mutation (TM).

In some aspects, the Fc region of the bispecific binding protein is aglycosylated. In some aspects, the Fc region of the bispecific binding protein is deglycosylated.

In some aspects, the Fc region of the bispecific binding protein has reduced fucosylation or is afucosylated.

In some aspects, the bispecific binding protein comprises a kappa light chain constant region. In some aspects, the bispecific binding protein comprises a lambda light chain constant region.

In some aspects, the bispecific binding protein is an antibody. In some aspects, the antibody is an IgG antibody. In some aspects, the antibody is an IgG1 antibody. In some aspects, the antibody is humanized.

In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer. In some aspects, the cancer is non-small cell lung cancer (NSCLC). In some aspects, the NSCLC is advanced or metastatic.

In some aspects, the subject has a PD-L1 tumor proportion score of greater than or equal to 1%. In some aspects, the subject has a PD-L1 tumor proportion score of greater than or equal to 50%. In some aspects, the subject is checkpoint inhibitor (CPI) naïve.

In some aspects, the present disclosure further provides a pharmaceutical composition comprising a bispecific binding protein that specifically binds to PD-1 and TIGIT in an amount from about 70 mg to about 1500 mg. In some aspects, the bispecific binding protein comprising: a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

In some aspects, the pharmaceutical composition comprises about 70 mg, about 150 mg, about 210 mg, about 450 mg, about 750 mg, about 800 mg, about 850 mg, about 900 mg, about 950 mg, about 1000 mg, about 1250 mg, or about 1500 mg bispecific binding protein. In some aspects, the pharmaceutical composition comprises about 750 mg bispecific binding protein. In some aspects, the pharmaceutical composition comprises about 1500 mg bispecific binding protein.

In some aspects where the cancer is NSCLC, the first binding domain of the bispecific binding protein that specifically binds to PD-1 comprises a heavy chain variable domain having the amino acid sequence of SEQ ID NO:7 and a light chain variable domain having the amino acid sequence of SEQ ID NO:9.

In some aspects, the first binding domain of the bispecific binding protein that specifically binds to PD-1 comprises a heavy chain variable domain having an amino acid sequence that is at least 90% identical to the amino acid sequence of SEQ ID NO:7 and a light chain variable domain having an amino acid sequence that is at least 90% identical to the amino acid sequence of SEQ ID NO:9.

In some aspects, the first binding domain of the bispecific binding protein that specifically binds to PD-1 comprises a heavy chain having the amino acid sequence of SEQ ID NO:8 and a light chain having the amino acid sequence of SEQ ID NO:10.

In some aspects, the first binding domain of the bispecific binding protein that specifically binds to PD-1 comprises a heavy chain having an amino acid sequence that is at least 90% identical to the amino acid sequence of SEQ ID NO:8 and a light chain having an amino acid sequence that is at least 90% identical to the amino acid sequence of SEQ ID NO:10.

In some aspects, the second binding domain of the bispecific binding protein that specifically binds to TIGIT comprises a heavy chain variable domain having the amino acid sequence of SEQ ID NO:17 and a light chain variable domain having the amino acid sequence of SEQ ID NO:19.

In some aspects, the second binding domain of the bispecific binding protein that specifically binds to TIGIT comprises a heavy chain variable domain having an amino acid sequence that is at least 90% identical to the amino acid sequence of SEQ ID NO:17 and a light chain variable domain having an amino acid sequence that is at least 90% identical to the amino acid sequence of SEQ ID NO:19.

In some aspects, the second binding domain of the bispecific binding protein that specifically binds to TIGIT comprises a heavy chain having the amino sequence of SEQ ID NO:18 and a light chain having the amino acid sequence of SEQ ID NO:20.

In some aspects, the second binding domain of the bispecific binding protein that specifically binds to TIGIT comprises a heavy chain having an amino acid sequence that is at least 90% identical to the amino acid sequence of SEQ ID NO:18 and a light chain having an amino acid sequence that is at least 90% identical to the amino acid sequence of SEQ ID NO:20.

The present disclosure further provides a kit comprising any of the pharmaceutical compositions described above. In some aspects, the kit further comprises instructions for administering the pharmaceutical composition.

The present disclosure further provides a pharmaceutical composition as defined above, for use in treating cancer.

In some aspects of the pharmaceutical composition for use, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer. In some aspects, the cancer is non-small cell lung cancer (NSCLC). In some aspects, the NSCLC is advanced or metastatic.

In some aspects of the pharmaceutical composition for use, the cancer is NSCLC and has a PD-L1 tumor proportion score of greater than or equal to 1%. In some aspects of the pharmaceutical composition for use, the cancer is NSCLC and has a PD-L1 tumor proportion score of greater than or equal to 50%.

In some aspects of the pharmaceutical composition for use, the cancer has not previously been treated with a checkpoint inhibitor.

### BRIEF DESCRIPTION OF THE DRAWINGS

The following drawings form part of the present specification and are included to further demonstrate exemplary aspects of the present disclosure.

FIG. 1 shows predicted PD-1 and TIGIT percentage receptor occupancy (RO) in Tumor. Trough concentration was used in prediction; the model assumes limited impact of ADA on PK. Low/high tumor PC=5%/50%, low/high tumoral PD-1 RC=3/30 nM, low/high tumoral TIGIT RC=4/40 nM. ADA=anti-drug antibodies; Ig=immunoglobulin; PC=partition coefficient; PD-1=programmed cell death-1; PK=pharmacokinetics; RC=receptor concentration; RO=receptor occupancy; TIGIT=T cell immunoreceptor with Ig and immunoreceptor tyrosine-based inhibitory motif domains.

FIG. 2 is a schematic diagram showing an example method of treatment comprising a dose escalation phase (Part A) and three dose expansion phases (Part B-Part D).

FIG. 3 is a chart of the adverse events occurring in greater than 5% of the 80 subjects evaluated in the Part A and Part B study described in Example 8 below. Adverse events related to AZD2936 are plotted on the right side of the chart.

FIG. 4 is a chart of the adverse events occurring in greater than 5% of the 54 subjects dosed with AZD2936 as evaluated in the Part A and Part B study described in Example 8 below. Adverse events related to AZD2936 are plotted on the right side of the chart.

FIG. 5 is a chart of the adverse events occurring in greater than 5% of the 83 subjects with AZD2936 as evaluated in the Part A and Part B expanded study described in Example 11 below. Treatment-emergent adverse events (TEAEs) are plotted on the left side of the chart, and treatment-related adverse events (TRAEs) are plotted on the right side of the chart.

FIG. 6 is a chart of the adverse events occurring in the 54 subjects receiving the recommended phase 2 dose (RP2D) of AZD2936 as evaluated in the Part A and Part B expanded study described in Example 11 below. TEAEs are plotted on the left side of the chart, and TRAEs are plotted on the right side of the chart.

FIG. 7 is a waterfall plot of post-baseline scans of 83 subjects as described in Example 11. Plots are for the best percentage change from baseline of the target lesion size.

FIG. 8 is a plot of the duration of exposure to AZD2936 for each subject measured in weeks for the result in FIG. 7.

FIGS. 9A-9C show pharmacodynamics of two subjects treated at the PR2D. FIG. 9A and FIG. 9B are radiographic responses of the first and second subjects at 9 weeks, respectively. FIG. 9C is a plot of the two subjects showing 100% reduction in circulating tumor deoxyribonucleic acid (ctDNA) at 6 weeks. Abbreviations in FIGS. 9A-9C: ctDNA, circulating tumour DNA; LN, lymph node; mVAF, mean variant allele frequency; PD-L1, programmed cell death ligand-1; RP2D, recommended phase 2 dose; TL, target lesion.

FIG. 10 is a chart of the adverse events occurring in greater than 10% of 54 subjects dosed with AZD2936 750 mg Q3W in the Part A and Part B study described in Example 8 below. Adverse events related to AZD2936 are plotted on the right side of the chart.

FIG. 11 is a chart of the adverse events occurring in greater than 10% of the 19 subjects dosed with AZD2936 750 mg Q3W as evaluated in the Part C study described in Example 12 below. Adverse events related to AZD2936 are plotted on the right side of the chart.

FIG. 12 is a chart of the adverse events occurring in greater than 10% of the 13 subjects dosed with AZD2936 1500 mg Q3W as evaluated in the part D study described in Example 13 below. Adverse events related to AZD2936 are plotted on the right side of the chart.

FIG. 13 is a chart of the adverse events occurring in greater than 10% of 14 subjects dosed with AZD2936 750 mgQ3W in Part D study described in Example 13 below. Adverse events related to AZD2936 are plotted on the right side of the chart.

FIG. 14 is a chart of the best percentage change from baseline in target lesion size in 19 subjects dosed with AZD2936 750 mg and 1500 mg Q3W as evaluated in Part C and D study described in Example 12 and Example 13 below. Each bar corresponds to one subject and is color coded to reflect the best overall response.

FIG. 15 is a chart of change of target lesion size over time in 19 subjects dosed with AZD2936 750 mg and 1500 mg Q3W as evaluated in Part C and D study described in Example 12 and Example 13 below. Each line represents corresponds to subject and is color coded to reflect the best overall response.

### DETAILED DESCRIPTION

### Definitions

Unless otherwise defined herein, scientific, and technical terms used in the present disclosure shall have the meanings that are commonly understood by one of ordinary skill in the art. Further, unless otherwise required by context, singular terms shall include pluralities and plural terms shall include the singular.

As used herein, “a” or “an” may mean one or more. As used herein, when used in conjunction with the word “comprising,” the words “a” or “an” may mean one or more than one. As used herein, “another” or “a further” may mean at least a second or more.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In some aspects, one of skill in the art will understand the level of variability indicated by the term “about,” due to the context in which it is used herein. It should also be understood that use of the term “about” also includes the specifically recited value.

The use of the term “for example” and its corresponding abbreviation “e.g.,” (whether italicized or not) means that the specific terms recited are representative examples and aspects of the disclosure that are not intended to be limited to the specific examples referenced or cited unless explicitly stated otherwise.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Those of skill in the art, however, would appreciate that the locations of the domains in a naturally occurring antibody can be modified in certain antibody-like binding protein formats without a loss of antigen-binding capability. Classes of human light chains are termed kappa and lambda light chains.

In some aspects, the light chain constant region is a kappa chain. In some aspects, light chain constant region is a lambda chain.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antibody fragment” refers to a portion of an intact or full-length chain or an antibody, generally the target binding or variable region. Examples of antibody fragments include, but are not limited to, F_{ab}, F_{ab′}, F_{(ab′)2} and Fv fragments. As used herein, the term “functional fragment” is generally synonymous with “antibody fragment,” and with respect to antibodies, can refer to antibody fragments such as Fᵥ, F_{ab}, F_{(ab′)2}.

Reference to the numbering of amino acid residues described herein is performed according to the EU numbering system (also described in Kabat et al, Sequences of Proteins of Immunological Interest, 5th Ed. Public Health Service, National Institutes of Health, Bethesda, MD. (1991)).

The term “human antibody,” as used herein, includes antibodies having variable and constant regions substantially corresponding to human germline immunoglobulin sequences. In some aspects, human antibodies are produced in non-human mammals, including, but not limited to, rodents, such as mice and rats, and lagomorphs, such as rabbits. In other aspects, human antibodies are produced in hybridoma cells. In still other aspects, human antibodies are produced recombinantly. In some aspects, the bispecific binding protein is a human or humanized antibody.

The term “antigen” or “target antigen” as used herein refers to a molecule or a portion of a molecule that is capable of being recognized by and bound by binding proteins of the disclosure. The target antigen is capable of being used in an animal to produce antibodies capable of binding to an epitope of that antigen. A target antigen may have one or more epitopes.

The term “epitope” as used herein refers to a region or structural element of an antigen that is recognized and bound by a binding protein of the disclosure. More precisely, the epitope is the specific structure that is bound by the CDRs of the binding protein. Epitopes can comprise protein structural elements, carbohydrates or even portions of lipid structures found in membranes. A binding protein is said to specifically bind an antigen when it preferentially recognizes its antigen target in a complex mixture of proteins and/or macromolecules. The term “specifically binds” refers to a binding protein that specifically binds to a molecule or a fragment thereof (e.g., antigen). A binding protein that specifically binds a molecule or a fragment thereof may bind to other molecules with lower affinity as determined by, for example, immunoassays, BIAcore, or other assays known in the art. In particular, antibodies or fragments that specifically bind to at least one molecule or a fragment thereof can compete off molecules that bind non-specifically. The present disclosure specifically encompasses antibodies with multiple specificities (e.g., an antibody with specificity for two or more discrete antigens. For example, a bispecific antibody can bind to two adjacent epitopes on a single target antigen, or can bind to two different antigens.

The term “native Fc” as used herein refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms.

The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc domain” as used herein encompasses native Fc and Fc variants and sequences as defined above. As with Fc variants and native Fc molecules, the term “Fc domain” includes molecules in monomeric or multimeric form, whether digested from whole antibody or produced by other means.

The term “treating” or “treatment” refers to administering a compound or pharmaceutical composition to a subject in order to effect an alteration or improvement of a disease, disorder, or condition in the subject. The terms “treatment” or “treat” as used herein may refer to both therapeutic treatment and prophylactic or preventative measures. Those in need of treatment include subjects having a disease or condition as well as those prone to having disease or condition or those for which a disease or condition is to be prevented.

The term “dose” means a specified quantity of a compound or pharmaceutical agent provided in a single administration, or in a specified time period. In some aspects, a dose can be administered in two or more boluses, tablets, or injections. For example, in some aspects, where subcutaneous administration is desired, the desired dose may require a volume not easily accommodated by a single injection. In such aspects, two or more injections can be used to achieve the desired dose. In some aspects, a dose can be administered in two or more injections to minimize injection site reaction in an individual. In other aspects, the compound or pharmaceutical agent is administered by infusion over an extended period of time or continuously. Doses can be stated as the amount of pharmaceutical agent per hour, day, week or month.

The terms “subject”, “individual” and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In a preferable aspect, the subject is a human. In methods of the invention, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, in one aspect, a method of the invention can be used to confirm the presence of cancer in a subject. For example, the subject may previously have been diagnosed with cancer by alternative means. In one aspect, the subject has been previously administered a cancer therapy. A subject who is “checkpoint inhibitor naïve” or “CPI naïve” means a subject whose cancer has not previously been treated with a checkpoint inhibitor (CPI).

The term “efficacy” means the ability to produce a desired effect. A “therapeutically effective dose” or “therapeutic dose” is an amount sufficient to effect desired clinical results (i.e., achieve therapeutic efficacy). A therapeutically effective dose can be administered in one or more administrations.

The term “side effects” means physiological disease and/or conditions attributable to a treatment other than the desired effects. In some aspects, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality. A “disease” or “condition” refers to any condition that would benefit from treatment using the methods of the disclosure. “Disease” and “condition” are used interchangeably herein and include chronic and acute disorders or diseases, including those pathological conditions that predispose a patient to the disorder in question. In some aspects, the disease is a tumor. In some aspects, the disease is a solid tumor. In some aspects, the disease is cancer. In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer. In some aspects, the disease is non-small cell lung cancer (NSCLC). In some aspects, the NSCLC is advanced or metastatic. In some aspects, the advanced NSCLC is stage III or stage IV NSCLC.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition” as used herein refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure.

The terms “pharmaceutically acceptable carrier” or “physiologically acceptable carrier” as used herein refer to one or more formulation materials suitable for accomplishing or enhancing the delivery of one or more binding proteins of the disclosure.

### Methods of Treating Cancer

The disclosure relates to a method for treating a cancer in a subject comprising administering to the subject a bispecific binding proteins that specifically binds to PD-1 and TIGIT in an amount from about 70 mg to about 1500 mg. The disclosure also provides compositions, including pharmaceutical compositions, and kits, comprising such bispecific protein.

As used herein, bispecific binding proteins have binding specificities for at least two independent antigens (or targets) or different epitopes within the same antigen. Exemplary bispecific binding proteins may bind to two different epitopes of a target, or may bind two different targets. Other such binding proteins may combine a first target binding site with a second binding site for another target. In some aspects, the binding protein is a bispecific antibody.

In some aspects, bispecific antibodies provide additive and/or synergistic therapeutic effects derived from targeting two antigens simultaneously, with the administration of a single manufactured molecule.

In some aspects, the antibodies provided herein are monovalent bispecific antibodies (MBab). The monovalent bispecific antibody scaffolds described herein provide a superior platform for the generation of bispecific antibodies that fulfill all the benefits associated with bispecific antibodies while reducing the potential therapeutic risks mentioned above due to their monovalent nature. Furthermore, the MBabs provided herein are readily expressed, stable, and are likely to have low immunogenicity. As used herein, the term “monovalent bispecific,” which can be abbreviated “MBab,” refers to bispecific antibodies, where each arm can specifically bind to a different target antigen, and for a given pair of different target antigens (A and B), the MBab can bind to one of each. In certain aspects, monovalent bispecific antibodies can specifically bind to two independent antigens (or targets) or two independent epitopes on the same antigen. Typically, monovalent bispecific antibodies comprise two different variable regions. In some aspects, the binding affinity for the two independent antigens is about the same. In some aspects, the binding affinities for the two independent antigens are different.

In some aspects, the bispecific binding protein comprises: a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

In some aspects, the first binding domain that specifically binds to PD-1 comprises a heavy chain variable domain having the amino acid sequence of SEQ ID NO:7 and a light chain variable domain having the amino acid sequence of SEQ ID NO:9. In some aspects, the first binding domain that specifically binds to PD-1 comprises a heavy chain having the amino acid sequence of SEQ ID NO:8 and a light chain having the amino acid sequence of SEQ ID NO:10.

In some aspects, the first binding domain that specifically binds to PD-1 comprises a heavy chain variable domain having an amino acid sequence that is at least 90, 91, 92, 93, 94, 95, 96, 97, 98, or 99% identical to the amino acid sequence of SEQ ID NO: 7. In some aspects, the first binding domain that specifically binds to PD-1 comprises a light chain variable domain having an amino acid sequence that is at least 90, 91, 92, 93, 94, 95, 96, 97, 98, or 99% identical to the amino acid sequence of SEQ ID NO: 9.

In some aspects, the first binding domain that specifically binds to PD-1 comprises a heavy chain having an amino acid sequence that is at least 90, 91, 92, 93, 94, 95, 96, 97, 98, or 99% identical to the amino acid sequence of SEQ ID NO: 8. In some aspects, the first binding domain that specifically binds to PD-1 comprises a light chain having an amino acid sequence that is at least 90, 91, 92, 93, 94, 95, 96, 97, 98, or 99% identical to the amino acid sequence of SEQ ID NO: 10.

In some aspects, the second binding domain that specifically binds to TIGIT comprises a heavy chain variable domain having the amino acid sequence of SEQ ID NO:17 and a light chain variable domain having the amino acid sequence of SEQ ID NO:19. In some aspects, the second binding domain that specifically binds to TIGIT comprises a heavy chain having the amino sequence of SEQ ID NO:18 and a light chain having the amino acid sequence of SEQ ID NO:20.

In some aspects, the second binding domain that specifically binds to TIGIT comprises a heavy chain variable domain having an amino acid sequence that is at least 90, 91, 92, 93, 94, 95, 96, 97, 98, or 99% identical to the amino acid sequence of SEQ ID NO: 17. In some aspects, the second binding domain that specifically binds to TIGIT comprises a light chain variable domain having an amino acid sequence that is at least 90, 91, 92, 93, 94, 95, 96, 97, 98, or 99% identical to the amino acid sequence of SEQ ID NO: 19.

In some aspects, the second binding domain that specifically binds to TIGIT comprises a heavy chain having an amino acid sequence that is at least 90, 91, 92, 93, 94, 95, 96, 97, 98, or 99% identical to the amino acid sequence of SEQ ID NO: 18. In some aspects, the second binding domain that specifically binds to TIGIT comprises a light chain having an amino acid sequence that is at least 90, 91, 92, 93, 94, 95, 96, 97, 98, or 99% identical to the amino acid sequence of SEQ ID NO: 20.

In some aspects, the first binding domain that specifically binds to PD-1 comprises a heavy chain variable domain that is encoded by the nucleic acid sequence of SEQ ID NO: 21 and a light chain variable domain is encoded by the nucleic acid sequence of SEQ ID NO: 23. In some aspects, the first binding domain that specifically binds to PD-1 comprises a heavy chain is encoded by the nucleic acid sequence of SEQ ID NO: 22 and a light chain is encoded by the nucleic acid sequence of SEQ ID NO: 24.

In some aspects, the first binding domain that specifically binds to PD-1 comprises a heavy chain variable domain that is encoded by a nucleic acid sequence that is at least 90, 91, 92, 93, 94, 95, 96, 97, 98, or 99% identical to SEQ ID NO: 21 and a light chain variable domain is encoded by a nucleic acid sequence that is at least 90, 91, 92, 93, 94, 95, 96, 97, 98, or 99% identical to SEQ ID NO: 23.

In some aspects, the first binding domain that specifically binds to PD-1 comprises a heavy chain is encoded by a nucleic acid sequence that is at least 90, 91, 92, 93, 94, 95, 96, 97, 98, or 99% identical to SEQ ID NO: 22 and a light chain is encoded by a nucleic acid sequence that is at least 90, 91, 92, 93, 94, 95, 96, 97, 98, or 99% identical to SEQ ID NO: 24.

In some aspects, the second binding domain that specifically binds TIGIT comprises a heavy chain variable domain that is encoded by the nucleic acid sequence of SEQ ID NO: 25 and a light chain variable domain that is encoded by the nucleic acid sequence of SEQ ID NO: 27. In some aspects, the second binding domain that specifically binds to TIGIT comprises a heavy chain that is encoded by the nucleic acid sequence of SEQ ID NO: 26 and a light chain that is encoded by the nucleic acid sequence of SEQ ID NO: 28.

In some aspects, the second binding domain that specifically binds TIGIT comprises a heavy chain variable domain that is encoded by a nucleic acid sequence that is at least 90, 91, 92, 93, 94, 95, 96, 97, 98, or 99% identical to SEQ ID NO: 25 and a light chain variable domain that is encoded by a nucleic acid sequence that is at least 90, 91, 92, 93, 94, 95, 96, 97, 98, or 99% identical to ID NO: 27.

In some aspects, the second binding domain that specifically binds to TIGIT comprises a heavy chain that is encoded by a nucleic acid sequence that is at least 90, 91, 92, 93, 94, 95, 96, 97, 98, or 99% identical to SEQ ID NO: 26 and a light chain that is encoded by a nucleic acid sequence that is at least 90, 91, 92, 93, 94, 95, 96, 97, 98, or 99% identical to SEQ ID NO: 28.

In some aspects, the Fc region is or includes a domain that is one or more of an Fc region from an IgG1, IgG2, IgG3, IgG4, IgA, IgM, IgE, or IgD. In some aspects, the antibody is an IgG1 antibody. In some aspects, the antibody is an IgG2 antibody. In some aspects, the antibody is an IgG3 antibody. In some aspects, the antibody is an IgG4 antibody.

In some aspects, the bispecific binding protein comprises a variant Fc region. Fc region engineering is widely used in the art to extend the half-life of therapeutic antibodies and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolism and protects IgG molecules from degradation.

In some aspects, the variant Fc region of the bispecific binding protein comprises at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 262I, 262A, 262T, 262E, 263I, 263A, 263T, 263M, 264L, 264I, 264W, 264T, 264R, 264F, 264M, 264Y, 264E, 265A, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

In some aspects, the variant Fc region comprises one or more modifications at positions selected from 428 and 434 as numbered by the EU index as set forth in Kabat. In some aspects, the variant Fc region comprises one or more amino acid substitutions at positions selected from 428 and 434 as numbered by the EU index as set forth in Kabat. In some aspects, the variant Fc region comprises one or more amino acid substitutions selected from 428L, 428F, 434A, 424F, 434W, and 434Y.

In some aspects, the variant Fc region of the bispecific binding protein comprises one or more amino acid substitutions at positions selected from 428 and 434 as numbered by the EU index as set forth in Kabat. In some aspects, the variant Fc region of the bispecific binding protein comprises one or more amino acid substitutions selected from 428L, 428F, 434A, 424F, 434W, and 434Y. In some aspects, the variant Fc region of the bispecific binding protein comprises a YTE mutation (M252Y/S254T/T256E).

In some aspects, the Fc variant antibody or binding fragment thereof has reduced antibody dependent cellular cytotoxicity (ADCC) when administered in vivo. In some aspects, the Fc variant antibody or binding fragment thereof has reduced ADCC compared to an antibody or binding variant thereof that contains a wild-type Fc region. In some aspects, the Fc variant antibody or binding fragment thereof does not trigger ADCC when administered in vivo. In some aspects, the Fc variant antibody or binding fragment thereof causes reduced ADCC when administered in vivo. In some aspects, the Fc variant antibody or binding fragment thereof having reduced ADCC activity or no ADCC activity comprises the L234F/L235E/P331S triple mutation (TM) in the variant Fc region.

In some aspects, the antibody or binding fragment thereof having reduced CDC activity has reduced toxicity when administered to a subject. In some aspects, the antibody or binding fragment thereof having reduced ADCC activity has reduced toxicity when administered to a subject.

In some aspects, the Fc region of the bispecific binding protein is aglycosylated. In some aspects, the Fc region of the bispecific binding protein is deglycosylated. In some aspects, the Fc region of the bispecific binding protein has reduced fucosylation or is afucosylated.

In some aspects, the bispecific binding protein comprises a kappa light chain constant region. In some aspects, the bispecific binding protein comprises a lambda light chain constant region.

In some aspects, the bispecific binding protein is an antibody. In some aspects, the antibody is an IgG antibody. In some aspects, the antibody is an IgG1 antibody. In some aspects, the antibody is an IgG2 antibody. In some aspects, the antibody is an IgG3 antibody. In some aspects, the antibody is an IgG4 antibody. In some aspects, the antibody is humanized.

In particular aspects, the disclosure provides methods of inducing an immune response in a subject as well as methods for treating or preventing a tumor and/or cancer in a subject by administering the proteins, nucleic acid molecules and/or compositions to the subject.

In particular aspects, provided herein is a method of inducing an immune response in a subject comprising administering to the subject a bispecific protein as described herein. In one aspect, provided herein is a method of inducing an immune response in a subject comprising administering to the subject a nucleic acid as described herein. In one aspect, provided herein is a method of inducing an immune response in a subject comprising administering to the subject a pharmaceutical composition as described herein.

In one aspect, provided herein is a bispecific protein as defined herein for use in therapy. In one aspect, provided herein is a bispecific protein as defined herein for use in the treatment of cancer.

In one aspect, provided herein is the use of a bispecific protein as defined herein in the manufacture of a medicament for the treatment of cancer.

In one aspect, provided herein is a nucleic acid as defined herein for use in therapy. In one aspect, provided herein is a nucleic acid as defined herein for use in the treatment of cancer.

In one aspect, provided herein is the use of a nucleic acid as defined herein in the manufacture of a medicament for the treatment of cancer.

In some aspects, the binding proteins disclosed herein can be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In some aspects, the amount of bispecific binding protein administered is about 50 mg to about 2000 mg. In some aspects, the amount of bispecific binding protein administered is about 70 mg to about 1500 mg. In some aspects, the amount of bispecific binding protein administered is about 100 mg to about 1400 mg. In some aspects, the amount of bispecific binding protein administered is about 200 mg to about 1250 mg. In some aspects, the amount of bispecific binding protein administered is about 500 mg to about 1000 mg. In some aspects, the amount of bispecific binding protein administered is about 600 mg to about 900 mg. In some aspects, the amount of bispecific binding protein administered is about 700 mg to about 800 mg.

In some aspects, the amount of bispecific binding protein administered is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 725 mg, about 750 mg, about 775 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1440 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some aspects, the amount of bispecific binding protein administered is about 70 mg, about 150 mg, about 210 mg, about 450 mg, about 750 mg, about 800 mg, about 850 mg, about 900 mg, about 950 mg, about 1000 mg, about 1250 mg, or about 1500 mg.

In some aspects, the amount of bispecific binding protein administered is about 750 mg. In some aspects, the amount of bispecific binding protein administered is about 1500 mg.

In some aspects, the amount of bispecific binding protein administered is 70 mg, 150 mg, 210 mg, 450 mg, 750 mg, 800 mg, 850 mg, 900 mg, 950 mg, 1000 mg, 1250 mg, or 1500 mg.

In some aspects, the amount of bispecific binding protein administered is 750 mg. In some aspects, the amount of bispecific binding protein administered is 1500 mg.

In some aspects, the bispecific binding protein is administered once per treatment cycle. In some aspects, the bispecific binding protein is administered twice per treatment cycle. In some aspects, the bispecific binding protein is administered three times per treatment cycle.

In some aspects, the treatment cycle is about 7 days, 14 days, 21 days, 28 days, or 35 days. In some aspects, the treatment cycle is about 7 days. In some aspects, the treatment cycle is about 21 days.

In some aspects, the treatment cycle is repeated for up to about 10 to about 100 cycles. In some aspects, the treatment cycle is repeated for up to about 20 to about 50 cycles. In some aspects, the treatment cycle is repeated for up to about 30 to about 40 cycles. In some aspects, the treatment cycle is repeated for up to 10, 15, 20, 25, 30, 35, 40, 45 or 50 cycles. In some aspects, the treatment cycle is repeated for up to 35 cycles.

In some aspects, the bispecific binding protein is administered to the subject as a monotherapy or a combinational therapy. In some aspects, the bispecific binding protein is administered to the subject as a monotherapy.

In one aspect, the method comprises administering to the subject a therapeutically effective amount of the binding proteins disclosed herein in combination with an additional anti-cancer compound. In some aspects, the anti-cancer compound is a small molecule drug. In some aspects, the anti-cancer compound is pemetrexed, carboplatin, gemcitabine, cisplatin, paclitaxel or combinations thereof. In some aspects, the binding protein and additional anti-cancer treatment are administered simultaneously. In some aspects, the binding protein and additional anti-cancer treatment are not administered simultaneously but are administered during the same treatment cycle.

In some aspects, the bispecific binding protein is suitable for oral administration, or parenteral administration, such as subcutaneous, intravenous, or intramuscular injection or infusion. In some aspects, the bispecific binding protein is administered by an intravenous infusion (IV).

In some aspects, the subject has not received a prior line of systemic therapy. In some aspects, the subject has previously received a chemotherapy. In some aspects, the chemotherapy includes a platinum-based chemotherapy. In some aspects, the prior line of systematic therapy includes checkpoint inhibitor (CPI) therapy.

In some aspects, the cancer comprises a cancer cell which expresses PD-L1. In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer.

In some aspects, the cancer is non-small cell lung cancer (NSCLC). In some aspects, the NSCLC is advanced or metastatic.

In aspects where the cancer is NSCLC, the subject has a PD-L1 tumor proportion score of greater than or equal to 1%. In some aspects where the cancer is NSCLC, the subject has a PD-L1 tumor proportion score of greater than or equal to 50%.

In some aspects, the subject is checkpoint inhibitor (CPI) naïve, meaning that that the subject has not previously been administered a CPI.

### Pharmaceutical Compositions

In some aspects, the present disclosure further provides a pharmaceutical composition comprising a bispecific binding protein that specifically binds to PD-1 and TIGIT in an amount from about 70 mg to about 1500 mg. In some aspects, the bispecific binding protein comprising: a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

In some aspects, the amount of bispecific binding protein administered is about 50 mg to about 2000 mg. In some aspects, the amount of bispecific binding protein administered is about 70 mg to about 1500 mg. In some aspects, the amount of bispecific binding protein administered is about 100 mg to about 1400 mg. In some aspects, the amount of bispecific binding protein administered is about 200 mg to about 1250 mg. In some aspects, the amount of bispecific binding protein administered is about 500 mg to about 1000 mg. In some aspects, the amount of bispecific binding protein administered is about 600 mg to about 900 mg. In some aspects, the amount of bispecific binding protein administered is about 700 mg to about 800 mg.

In some aspects, the amount of bispecific binding protein administered is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 725 mg, about 750 mg, about 775 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1440 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some aspects, the amount of bispecific binding protein administered is about 70 mg, about 150 mg, about 210 mg, about 450 mg, about 750 mg, about 800 mg, about 850 mg, about 900 mg, about 950 mg, about 1000 mg, about 1250 mg, or about 1500 mg.

In some aspects, the amount of bispecific binding protein administered is about 750 mg. In some aspects, the amount of bispecific binding protein administered is about 1500 mg.

In some aspects, the amount of bispecific binding protein administered is 70 mg, 150 mg, 210 mg, 450 mg, 750 mg, 800 mg, 850 mg, 900 mg, 950 mg, 1000 mg, 1250 mg, or 1500 mg.

In some aspects, the amount of bispecific binding protein administered is 750 mg. In some aspects, the amount of bispecific binding protein administered is 1500 mg.

In some aspects, the first binding domain of the bispecific binding protein that specifically binds to PD-1 comprises a heavy chain variable domain having the amino acid sequence of SEQ ID NO:7 and a light chain variable domain having the amino acid sequence of SEQ ID NO:9. In some aspects, the first binding domain of the bispecific binding protein that specifically binds to PD-1 comprises a heavy chain variable domain having an amino acid sequence that is at least 90% identical to the amino acid sequence of SEQ ID NO:7 and a light chain variable domain having an amino acid sequence that is at least 90% identical to the amino acid sequence of SEQ ID NO:9.

In some aspects, the first binding domain of the bispecific binding protein that specifically binds to PD-1 comprises a heavy chain having the amino acid sequence of SEQ ID NO:8 and a light chain having the amino acid sequence of SEQ ID NO:10. In some aspects, the first binding domain of the bispecific binding protein that specifically binds to PD-1 comprises a heavy chain having an amino acid sequence that is at least 90% identical to the amino acid sequence of SEQ ID NO:8 and a light chain having an amino acid sequence that is at least 90% identical to the amino acid sequence of SEQ ID NO:10.

In some aspects, the second binding domain of the bispecific binding protein that specifically binds to TIGIT comprises a heavy chain variable domain having the amino acid sequence of SEQ ID NO:17 and a light chain variable domain having the amino acid sequence of SEQ ID NO:19. In some aspects, the second binding domain of the bispecific binding protein that specifically binds to TIGIT comprises a heavy chain variable domain having an amino acid sequence that is at least 90% identical to the amino acid sequence of SEQ ID NO:17 and a light chain variable domain having an amino acid sequence that is at least 90% identical to the amino acid sequence of SEQ ID NO:19.

In some aspects, the second binding domain of the bispecific binding protein that specifically binds to TIGIT comprises a heavy chain having the amino sequence of SEQ ID NO:18 and a light chain having the amino acid sequence of SEQ ID NO:20. In some aspects, the second binding domain of the bispecific binding protein that specifically binds to TIGIT comprises a heavy chain having an amino acid sequence that is at least 90% identical to the amino acid sequence of SEQ ID NO:18 and a light chain having an amino acid sequence that is at least 90% identical to the amino acid sequence of SEQ ID NO:20.

In some aspects, the pharmaceutical compositions disclosed herein can be formulated with a pharmaceutically acceptable carrier, excipient, or stabilizer. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which can be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

The present disclosure further provides a pharmaceutical composition as defined above, for use in treating cancer. In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer. In some aspects, the cancer is non-small cell lung cancer (NSCLC). In some aspects, the NSCLC is advanced or metastatic.

### Kits

In some aspects, the present disclosure further provides a kit comprising any of the pharmaceutical compositions described above. In some aspects, the kit comprises instructions for administering the pharmaceutical composition. In some aspects, the kit comprises an additional anti-cancer agent as described herein.

### Bispecific Binding Molecule Sequences

Aspects of the disclosure include bispecific binding proteins in DuetMab format that bind PD-1 and TIGIT created using the sequences in Table 1 below. CDRs in Table 1 are determined based on the system in Kabat.

TABLE 1
Sequences for PD-1/TIGIT Bispecific Binding Molecules

| | | |
|---|---|---|
| PD1 LO115 | SEQ ID | DYGMH |
| HCDR1 | NO: 1 | |
| PD1 LO115 | SEQ ID | YISSGSYTIYSADSVKG |
| HCDR2 | NO: 2 | |
| PD1 LO115 | SEQ ID | RAPNSFYEYYFDY |
| HCDR3 | NO: 3 | |
| PD1 LO115 | SEQ ID | SASSKHTNLYWSRHMY |
| LCDR1 | NO: 4 | |
| PD1 LO115 | SEQ ID | LTSNRAT |
| LCDR2 | NO: 5 | |
| PD1 LO115 | SEQ ID | QQWSSNPFT |
| LCDR3 | NO: 6 | |
| PD1 LO115 | SEQ ID | EVQLVESGGGLVQPGGSLRLSCAASGFTFSDYGMHWVR |
| variable | NO: 7 | QAPGKGLEWVAYISSGSYTIYSADSVKGRFTISRDNAK |
| heavy chain | | NSLYLQMNSLRAEDTAVYYCARRAPNSFYEYYFDYWGQ |
| | | GTTVTVSS |
| PD1 LO115 | SEQ ID | EVQLVESGGGLVQPGGSLRLSCAASGFTFSDYGMHWVR |
| Hole heavy | NO: 8 | QAPGKGLEWVAYISSGSYTIYSADSVKGRFTISRDNAK |
| chain | | NSLYLQMNSLRAEDTAVYYCARRAPNSFYEYYFDYWGQ |
| | | GTTVTVSSASTKGPSVFPLAPSSKSTSGGTAALGCLVK |
| | | DYFPEPVTVSWNSGALTSGVHTFPAVLQSSGLYSLSSV |
| | | VTVPSSSLGTQTYICNVNHKPSNTKVDKRVEPKSCDKT |
| | | HTCPPCPAPEFEGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPASIEKTIS |
| | | KAKGQPREPQVCTLPPSREEMTKNQVSLSCAVKGFYPS |
| | | DIAVEWESNGQPENNYKTTPPVLDSDGSFFLVSKLTVD |
| | | KSRWQQGNVFSCSVMHEALHNHYTQKSLSLSPGK |
| PD1 LO115 | SEQ ID | QIVLTQSPATLSLSPGERATLSCSASSKHTNLYWSRHM |
| variable | NO: 9 | YWYQQKPGQAPRLLIYLTSNRATGIPARFSGSGSGTDF |
| light chain | | TLTISSLEPEDFAVYYCQQWSSNPFTFGQGTKLEIK |
| PD1 LO115 | SEQ ID | QIVLTQSPATLSLSPGERATLSCSASSKHTNLYWSRHM |
| WT Kappa | NO: 10 | YWYQQKPGQAPRLLIYLTSNRATGIPARFSGSGSGTDF |
| light chain | | TLTISSLEPEDFAVYYCQQWSSNPFTFGQGTKLEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |
| TIGIT | SEQ ID | SYAMH |
| HCDR1 | NO: 11 | |
| TIGIT | SEQ ID | VISYAGEVKYYADSVKG |
| HCDR2 | NO: 12 | |
| TIGIT | SEQ ID | DPLPLHYYGMDV |
| HCDR3 | NO: 13 | |
| TIGIT | SEQ ID | SGSSSNIGRRPVN |
| LCDR1 | NO: 14 | |
| TIGIT | SEQ ID | SQNQRPS |
| LCDR2 | NO: 15 | |
| TIGIT | SEQ ID | AVWDDIGRVLQ |
| LCDR3 | NO: 16 | |
| TIGIT | SEQ ID | QVQLVESGGGVVQPGRSLRLSCAASGFTFSSYAMHWVR |
| variable | NO: 17 | QAPGKGLEWVAVISYAGEVKYYADSVKGRFTISRDNSK |
| heavy chain | | NTLYLQMNSLRAEDTAVYYCARDPLPLHYYGMDVWGQG |
| | | TTVTVSS |
| TIGIT Knob | SEQ ID | QVQLVESGGGVVQPGRSLRLSCAASGFTFSSYAMHWVR |
| heavy chain | NO: 18 | QAPGKGLEWVAVISYAGEVKYYADSVKGRFTISRDNSK |
| | | NTLYLQMNSLRAEDTAVYYCARDPLPLHYYGMDVWGQG |
| | | TTVTVSSASTKGPSVCPLAPSSKSTSGGTAALGCLVKD |
| | | YFPEPVTVSWNSGALTSGVHTFPAVLQSSGLYSLSSVV |
| | | TVPSSSLGTQTYICNVNHKPSNTKVDKRVEPKSVDKTH |
| | | TCPPCPAPEFEGGPSVFLFPPKPKDTLMISRTPEVTCV |
| | | VVDVSHEDPEVKFNWYVDGVEVHNAKTKPREEQYNSTY |
| | | RVVSVLTVLHQDWLNGKEYKCKVSNKALPASIEKTISK |
| | | AKGQPREPQVYTLPPCREEMTKNQVSLWCLVKGFYPSD |
| | | IAVEWESNGQPENNYKTTPPVLDSDGSFFLYSKLTVDK |
| | | SRWQQGNVFSCSVMHEALHNHYTQKSLSLSPGK |
| TIGIT | SEQ ID | QSVLTQPPSASGTPGQRVTISCSGSSSNIGRRPVNWYQ |
| variable | NO: 19 | QLPGTAPKLLIYSQNQRPSGVPDRFSGSQSGTSASLAI |
| light chain | | SGLQSEDEADYFCAVWDDIGRVLQLGGGTQLTVL |
| TIGIT | SEQ ID | QSVLTQPPSASGTPGQRVTISCSGSSSNIGRRPVNWYQ |
| engineered | NO: 20 | QLPGTAPKLLIYSQNQRPSGVPDRFSGSQSGTSASLAI |
| Lambda | | SGLQSEDEADYFCAVWDDIGRVLQLGGGTQLTVLGQPK |
| light chain | | AAPSVTLFPPCSEELQANKATLVCLISDFYPGAVTVAW |
| | | KADSSPVKAGVETTTPSKQSNNKYAASSYLSLTPEQWK |
| | | SHRSYSCQVTHEGSTVEKTVAPTEVS |
| PD1 LO115 | SEQ ID | GAGGTGCAGCTGGTGGAATCCGGCGGAGGACTGGTGCA |
| variable | NO: 21 | GCCTGGCGGCTCCCTGAGACTGTCTTGCGCCGCCTCCG |
| heavy chain | | GCTTCACATTCTCCGACTACGGCATGCACTGGGTCCGA |
| | | CAGGCCCCTGGAAAGGGCCTGGAATGGGTGGCCTACAT |
| | | CTCCTCCGGCTCCTACACCATCTACTCCGCCGACTCCG |
| | | TGAAGGGCCGGTTCACCATCTCCCGGGACAACGCCAAG |
| | | AACTCCCTGTACCTGCAGATGAACTCCCTGCGGGCCGA |
| | | GGACACAGCCGTGTACTACTGTGCCAGACGGGCCCCTA |
| | | ACTCCTTCTACGAGTACTACTTCGACTACTGGGGCCAG |
| | | GGCACCACCGTGACCGTGTCCTCT |
| PD1 LO115 | SEQ ID | GAGGTGCAGCTGGTGGAATCCGGCGGAGGACTGGTGCA |
| Hole heavy | NO: 22 | GCCTGGCGGCTCCCTGAGACTGTCTTGCGCCGCCTCCG |
| chain | | GCTTCACATTCTCCGACTACGGCATGCACTGGGTCCGA |
| | | CAGGCCCCTGGAAAGGGCCTGGAATGGGTGGCCTACAT |
| | | CTCCTCCGGCTCCTACACCATCTACTCCGCCGACTCCG |
| | | TGAAGGGCCGGTTCACCATCTCCCGGGACAACGCCAAG |
| | | AACTCCCTGTACCTGCAGATGAACTCCCTGCGGGCCGA |
| | | GGACACAGCCGTGTACTACTGTGCCAGACGGGCCCCTA |
| | | ACTCCTTCTACGAGTACTACTTCGACTACTGGGGCCAG |
| | | GGCACCACCGTGACCGTGTCCTCTGCTAGCACCAAAGG |
| | | TCCGAGCGTTTTTCCGCTGGCACCGAGCAGCAAAAGCA |
| | | CCTCTGGTGGCACCGCAGCACTGGGTTGTCTGGTGAAA |
| | | GATTATTTTCCGGAACCGGTTACCGTTTCTTGGAATAG |
| | | CGGTGCACTGACCAGCGGTGTTCATACCTTTCCGGCAG |
| | | TTCTGCAGAGCAGCGGTCTGTATAGCCTGTCTAGCGTT |
| | | GTTACCGTTCCGAGCAGCAGCCTGGGCACCCAGACCTA |
| | | TATTTGCAATGTGAATCATAAACCGAGCAATACAAAAG |
| | | TTGATAAACGCGTTGAACCGAAAAGCTGTGACAAAACT |
| | | CACACGTGCCCACCGTGCCCAGCACCTGAGTTCGAGGG |
| | | GGGACCGTCAGTCTTCCTCTTCCCCCCAAAACCCAAGG |
| | | ACACCCTCATGATCTCCCGGACCCCTGAGGTCACATGC |
| | | GTGGTGGTGGACGTGAGCCACGAAGACCCTGAGGTCAA |
| | | GTTCAACTGGTACGTGGACGGCGTGGAGGTGCATAATG |
| | | CCAAGACAAAGCCGCGGGAGGAGCAGTACAACAGCACG |
| | | TACCGTGTGGTCAGCGTCCTCACCGTCCTGCACCAGGA |
| | | CTGGCTGAATGGCAAGGAGTACAAGTGCAAGGTCTCCA |
| | | ACAAAGCCCTCCCAGCCAGCATCGAGAAAACCATCTCC |
| | | AAAGCCAAAGGGCAGCCCCGAGAACCACAGGTCTGCAC |
| | | CCTGCCCCCATCCCGGGAGGAGATGACCAAGAACCAGG |
| | | TCAGCCTGAGCTGCGCGGTCAAAGGCTTCTATCCCAGC |
| | | GACATCGCCGTGGAGTGGGAGAGCAATGGGCAGCCGGA |
| | | GAACAACTACAAGACCACGCCTCCCGTGCTGGACTCCG |
| | | ACGGCTCCTTCTTCCTCGTTAGCAAGCTCACCGTGGAC |
| | | AAGAGCAGGTGGCAGCAGGGGAACGTCTTCTCATGCTC |
| | | CGTGATGCATGAGGCTCTGCACAACCACTACACGCAGA |
| | | AGAGCCTCTCCCTGTCTCCGGGTAAATGA |
| PD1 LO115 | SEQ ID | CAGATCGTGCTGACCCAGTCCCCTGCCACCCTGTCCCT |
| variable | NO: 23 | GAGCCCTGGCGAGAGAGCCACCCTGAGCTGCTCCGCCT |
| light chain | | CCTCCAAGCACACCAACCTGTACTGGTCCCGGCACATG |
| | | TACTGGTATCAGCAGAAGCCCGGCCAGGCCCCTCGGCT |
| | | GCTGATCTACCTGACCTCTAACCGGGCCACCGGCATCC |
| | | CTGCCAGATTCTCCGGCTCTGGCTCCGGCACCGACTTC |
| | | ACCCTGACCATCTCCAGCCTGGAACCCGAGGACTTCGC |
| | | CGTGTACTACTGCCAGCAGTGGTCCTCCAACCCCTTCA |
| | | CCTTCGGCCAGGGCACCAAGCTGGAAATCAAG |
| PD1 LO115 | SEQ ID | CAGATCGTGCTGACCCAGTCCCCTGCCACCCTGTCCCT |
| WT Kappa | NO: 24 | GAGCCCTGGCGAGAGAGCCACCCTGAGCTGCTCCGCCT |
| light chain | | CCTCCAAGCACACCAACCTGTACTGGTCCCGGCACATG |
| | | TACTGGTATCAGCAGAAGCCCGGCCAGGCCCCTCGGCT |
| | | GCTGATCTACCTGACCTCTAACCGGGCCACCGGCATCC |
| | | CTGCCAGATTCTCCGGCTCTGGCTCCGGCACCGACTTC |
| | | ACCCTGACCATCTCCAGCCTGGAACCCGAGGACTTCGC |
| | | CGTGTACTACTGCCAGCAGTGGTCCTCCAACCCCTTCA |
| | | CCTTCGGCCAGGGCACCAAGCTGGAAATCAAGCGTACG |
| | | GTGGCTGCACCATCTGTCTTCATCTTCCCGCCATCTGA |
| | | TGAGCAGTTGAAATCTGGAACTGCCTCTGTTGTGTGCC |
| | | TGCTGAATAACTTCTATCCCAGAGAGGCCAAAGTACAG |
| | | TGGAAGGTGGATAACGCCCTCCAATCGGGTAACTCCCA |
| | | GGAGAGTGTCACAGAGCAGGACAGCAAGGACAGCACCT |
| | | ACAGCCTCAGCAGCACCCTGACGCTGAGCAAAGCAGAC |
| | | TACGAGAAACACAAAGTCTACGCCTGCGAAGTCACCCA |
| | | TCAGGGCCTGAGCTCGCCCGTCACAAAGAGCTTCAACA |
| | | GGGGAGAGTGTTAG |
| TIGIT | SEQ ID | CAGGTGCAGCTGGTGGAGTCTGGAGGAGGCGTGGTCCA |
| variable | NO: 25 | GCCTGGGAGGTCCCTGAGACTCTCCTGTGCAGCCTCTG |
| heavy chain | | GATTCACCTTCAGTAGCTATGCTATGCACTGGGTCCGC |
| | | CAGGCTCCAGGCAAGGGGCTGGAGTGGGTGGCAGTTAT |
| | | ATCATATGCGGGGGAGGTGAAATACTACGCAGACTCCG |
| | | TGAAGGGCCGATTCACCATCTCCAGAGACAATTCCAAG |
| | | AACACGCTGTATCTGCAAATGAACAGCCTGAGAGCTGA |
| | | GGACACGGCTGTGTATTACTGTGCGAGAGACCCGCTAC |
| | | CGCTACATTACTACGGTATGGACGTCTGGGGCCAAGGG |
| | | ACCACGGTCACCGTCTCCTCA |
| TIGIT Knob | SEQ ID | CAGGTGCAGCTGGTGGAGTCTGGAGGAGGCGTGGTCCA |
| heavy chain | NO: 26 | GCCTGGGAGGTCCCTGAGACTCTCCTGTGCAGCCTCTG |
| | | GATTCACCTTCAGTAGCTATGCTATGCACTGGGTCCGC |
| | | CAGGCTCCAGGCAAGGGGCTGGAGTGGGTGGCAGTTAT |
| | | ATCATATGCGGGGGAGGTGAAATACTACGCAGACTCCG |
| | | TGAAGGGCCGATTCACCATCTCCAGAGACAATTCCAAG |
| | | AACACGCTGTATCTGCAAATGAACAGCCTGAGAGCTGA |
| | | GGACACGGCTGTGTATTACTGTGCGAGAGACCCGCTAC |
| | | CGCTACATTACTACGGTATGGACGTCTGGGGCCAAGGG |
| | | ACCACGGTCACCGTCTCCTCAGCGTCGACCAAAGGTCC |
| | | GAGCGTGTGCCCGCTGGCACCGAGCAGCAAAAGCACCT |
| | | CTGGTGGCACCGCAGCACTGGGTTGTCTGGTGAAAGAT |
| | | TATTTTCCGGAACCGGTTACCGTTTCTTGGAATAGCGG |
| | | TGCACTGACCAGCGGTGTTCATACCTTTCCGGCAGTCC |
| | | TGCAGAGCAGCGGTCTGTATAGCCTGTCTAGCGTTGTT |
| | | ACCGTTCCGAGCAGCAGCCTGGGCACCCAGACCTATAT |
| | | TTGCAATGTGAATCATAAACCGAGCAATACCAAAGTTG |
| | | ATAAACGCGTTGAACCGAAAAGCGTGGACAAAACTCAC |
| | | ACGTGCCCACCGTGCCCAGCACCTGAGTTCGAGGGGGG |
| | | ACCGTCAGTCTTCCTCTTCCCCCCAAAACCCAAGGACA |
| | | CCCTCATGATCTCCCGGACCCCTGAGGTCACATGCGTG |
| | | GTGGTGGACGTGAGCCACGAAGACCCTGAGGTCAAGTT |
| | | CAACTGGTACGTGGACGGCGTGGAGGTGCATAATGCCA |
| | | AGACAAAGCCGCGGGAGGAGCAGTACAACAGCACGTAC |
| | | CGTGTGGTCAGCGTCCTCACCGTCCTGCACCAGGACTG |
| | | GCTGAATGGCAAGGAGTACAAGTGCAAGGTCTCCAACA |
| | | AAGCCCTCCCAGCCAGCATCGAGAAAACCATCTCCAAA |
| | | GCCAAAGGGCAGCCCCGAGAACCACAGGTCTACACCCT |
| | | GCCCCCATGCCGGGAGGAGATGACCAAGAACCAGGTCA |
| | | GCCTGTGGTGCCTGGTCAAAGGCTTCTATCCCAGCGAC |
| | | ATCGCCGTGGAGTGGGAGAGCAATGGGCAGCCGGAGAA |
| | | CAACTACAAGACCACGCCTCCCGTGCTGGACTCCGACG |
| | | GCTCCTTCTTCCTCTATAGCAAGCTCACCGTGGACAAG |
| | | AGCAGGTGGCAGCAGGGGAACGTCTTCTCATGCTCCGT |
| | | GATGCATGAGGCTCTGCACAACCACTACACGCAGAAGA |
| | | GCTTAAGCCTGTCTCCGGGTAAATGA |
| TIGIT | SEQ ID | CAGTCTGTGCTGACTCAGCCTCCCTCAGCGTCTGGGAC |
| variable | NO: 27 | CCCCGGGCAGAGGGTCACCATCTCTTGTTCTGGAAGCA |
| light chain | | GCTCCAATATCGGAAGGAGGCCTGTAAACTGGTACCAG |
| | | CAGCTCCCAGGAACGGCCCCCAAACTCCTCATCTATAG |
| | | TCAGAATCAGCGGCCCTCAGGGGTCCCTGACCGATTCT |
| | | CTGGCTCCCAGTCTGGCACCTCAGCCTCCCTGGCCATC |
| | | AGTGGGCTCCAGTCTGAGGATGAGGCTGATTATTTCTG |
| | | TGCAGTTTGGGATGACATCGGCCGCGTCCTGCAGTTAG |
| | | GCGGAGGCACCCAGCTGACCGTCCTA |
| TIGIT | SEQ ID | CAGTCTGTGCTGACTCAGCCTCCCTCAGCGTCTGGGAC |
| engineered | NO: 28 | CCCCGGGCAGAGGGTCACCATCTCTTGTTCTGGAAGCA |
| Lambda | | GCTCCAATATCGGAAGGAGGCCTGTAAACTGGTACCAG |
| light chain | | CAGCTCCCAGGAACGGCCCCCAAACTCCTCATCTATAG |
| | | TCAGAATCAGCGGCCCTCAGGGGTCCCTGACCGATTCT |
| | | CTGGCTCCCAGTCTGGCACCTCAGCCTCCCTGGCCATC |
| | | AGTGGGCTCCAGTCTGAGGATGAGGCTGATTATTTCTG |
| | | TGCAGTTTGGGATGACATCGGCCGCGTCCTGCAGTTAG |
| | | GCGGAGGCACCCAGCTGACCGTCCTAGGTCAGCCCAAG |
| | | GCGGCCCCCTCGGTCACTCTGTTCCCGCCCTGCTCTGA |
| | | GGAGCTTCAAGCCAACAAGGCCACACTGGTGTGTCTCA |
| | | TAAGTGACTTCTACCCGGGAGCCGTGACAGTGGCCTGG |
| | | AAGGCAGATAGCAGCCCCGTCAAGGCGGGAGTGGAGAC |
| | | CACCACACCCTCCAAACAAAGCAACAACAAGTACGCGG |
| | | CCAGCAGCTACCTGAGCCTGACGCCTGAGCAGTGGAAG |
| | | TCCCACAGAAGCTACAGCTGCCAGGTCACGCATGAAGG |
| | | GAGCACCGTGGAGAAGACAGTGGCCCCTACAGAAGTGT |
| | | CATGA |

The use of an anti-TIGIT/anti-PD-1 bispecific antibody is an innovative approach for targeting both TIGIT/PD-1 receptors at the same time. This approach has several potential advantages as compared with the co-administration of separate anti-TIGIT and anti-PD-1/PD-L1 antibodies. Along with the ease of drug delivery (a single administration replacing 2 administrations), there is a certainty of equal biodistribution of a single molecule targeting both receptors. Furthermore, the design of a bispecific antibody allows two potentially different modes of action, one targeting both receptors in close proximity on the same cell (cis effect) and another targeting the two receptors on two neighboring cells with the generation of a prolonged immune synapse simultaneously with checkpoint inhibition (trans effects).

All references cited herein, including patents, patent applications, papers, textbooks and the like, and the references cited therein, to the extent that they are not already, are hereby incorporated herein by reference in their entirety.

Without limiting the disclosure, a number of aspects of the disclosure are described herein for purpose of illustration.

### EXAMPLES

The Examples that follow are illustrative of specific aspects of the disclosure, and various uses thereof. They are set forth for explanatory purposes only and should not be construed as limiting the scope of the disclosure in any way.

AZD2936 is provided for treatment in the Examples. AZD2936 is a monovalent, bispecific, humanized immunoglobulin G (IgG) 1 monoclonal antibody (mAb) with an engineered fragment crystallizable (Fc) domain to reduce Fc effector function (IgG1-triple mutation). AZD2936 specifically binds to human T cell immunoreceptor with immunoglobulin and immunoreceptor tyrosine based inhibitory motif domains (TIGIT) and programmed cell death protein 1 (PD-1), which are part of a complex system of cell surface receptors that, when bound to their cognate ligands, provide co-inhibitory signals to T cells to modulate their activity. In the preclinical studies, dual blockade of TIGIT and PD-1 by AZD2936 enhanced human T cell function and promoted antitumor immune responses. AZD2936 is used in the treatment of subjects with advanced or metastatic, non-small cell lung cancer (NSCLC) expressing programmed cell death-ligand 1 (PD-L1; tumor proportion score [TPS]≥1%). AZD2936 is described in International Patent Application Publication WO2022/229919, which is hereby incorporated by reference herein.

### Example 1: Predicting Receptor Occupancy of TIGIT and PD-1 to AZD2936 in Solid Tumor Patients

As described in the Examples below, AZD2936 monotherapy is being investigated at dose levels ranging from 70 to 1500 mg Q3W intravenously. High intra-tumoral receptor occupancy (RO) in the tumor is believed to be required for efficacy in patients. Model simulation can be used to predict the RO for PD-1 and TIGIT at different dose levels and support the recommended phase 2 dose (RP2D) and dose optimization approach for AZD2936.

The model was developed by connecting a two-compartment PK model representing clearance and transport of AZD2936 in central and peripheral compartment, a tumor compartment, and two independent target-mediated drug disposition (TMDD) modules in central and tumor compartment to capture the drug concentration and receptor binding dynamics. The two-compartment PK model parameters were estimated in a separate population PK analysis. The tumor compartment was considered as an effect compartment, which assumes the distribution of AZD2936 to the tumor does not affect its blood concentration.

Sensitivity analysis was performed to identify mechanisms that have significant impact on intra-tumoral RO of PD-1 and TIGIT. To predict RO among the patient population, simulations were performed by incorporating variability attributed to these mechanisms in addition to PK variabilities. Predicted RO was compared between different AZD2936 dose levels and against approved anti-PD-1 compounds.

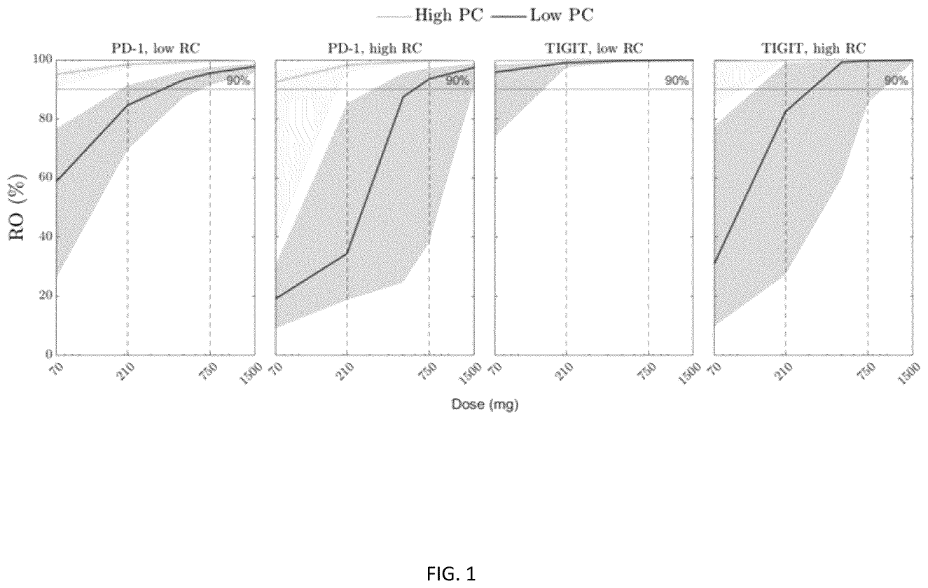

The model was developed by connecting a two-compartment PK model representing clearance and distribution of AZD2936 in central and peripheral compartment, a tumor compartment, and two independent target-mediated drug disposition (TMDD) modules in central and tumor compartment to capture the drug concentration and receptor binding dynamics. The tumor compartment was considered as an effect compartment, which assumes the distribution of AZD2936 to the tumor does not affect its blood concentration. The overall schematic of the PK/PD model is shown in FIG. 1. The model was then used to predict the receptor occupancy of PD-1 and TIGIT in both blood and tumor following IV administration of AZD2936 at various dose levels.

PD1 and TIGIT expressions were measured in peripheral blood mononuclear cells (PBMC). The concentrations of the four types of receptors were estimated from PBMC data. First, concentrations of T cell, NK cell, and other PBMC of PD1+TIGIT+/PD1+TIGIT−/PD1−TIGIT+receptor expression types were calculated. Analysis of TIGIT PD1 on PBMC in combination with prior results suggests that a similar number of PD1 or TIGIT are expressed on cells that co-express them (see Johnston et al., Cancer Cell, 26(6):923-937, 2014). The ratios of initial concentrations of PD1 and TIGIT on PD1+TIGIT+ cells (designated as RA₀ and RB₀), PD1 on PD1+TIGIT− cells (RC₀), and TIGIT on PD1−TIGIT+ cells (RD₀) were calculated to be 1:1:2:3.

Concentrations of receptors (RA₀, RB₀, RC₀, RD₀) in the central compartment were calculated by multiplying the concentrations of T cell, NK cell, and other PBMC of PD1+TIGIT+/PD1+TIGIT−/PD1−TIGIT+ by the number of receptors per cell, which is assumed to be between 10,000 and 1,000,000. The resulting receptor concentration for PD1 in PD1+TIGIT cells RA₀ is 0.001-0.1 nM. The other receptor concentrations (RB₀, RC₀, RD₀) were calculated using the aforementioned ratio of 1:1:2:3.

From the analysis of PBMCs, it can be derived that about 78% T cell are PD1−TIGIT− in blood PBMC. Assuming (i) this percentage is lower in the tumor micro-environment (TME) due to induction of PD1/TIGIT expression, (ii) more PD1 receptors per cell is induced in the TME compared with those in the blood (M. Mkrtichyan et al., J Immunol, 189(5): 2338-2347, 2012), and (iii) the ratio of PD1+TIGIT+, PD1+TIGIT−, and PD1−TIGIT+ T cells remains the same as in blood, the percentages of these cell types can be calculated. Total tumor T cell volume densities can be calculated from area densities (which can be measured through digital pathology from patient tumor samples) using the stereology formula from J.-P. Royet, Progress in Neurobiology, 37(5):433-474, 1991.

In the analysis dataset for AZD2936 there were 72 patients who received either 70 mg, 210 mg, 750 mg, or 1500 mg Q3W. The AZD2936 PK was reasonably described using 2 compartment model with first order elimination. Body weight was applied on PK using allometric scaling approach (0.75 on clearance of elimination (C_{L}) and intercompartmental clearance (Q); 1 on central volume of distribution (VC) and peripheral volume of distribution (VP)). Between-subject variability was estimated for C_{L}, VC and VP. The parameter estimates were C_{L}, 0.392 L/day; VC, 3.63 L; VP, 2.94 L; Q, 0.935 L/day, additive error 2.11 μg/mL, and proportional error 29.05%.

AZD2936 dosing was set to be scheduled Q3W as IV infusion. Dose levels for simulations were set to 70,210,500, 750 and 1500 mg. For each of the four combinations of partition coefficient (PC) and receptor concentration (RC), 1000 virtual patients are simulated.

Among the 1000 virtual patients, five parameters were varied in a latin hypercube sampling (LHS) sampling scheme: RA_{0,VC}, RA_{0,VT}, k_{int,PD}1, k_{eq,AZD2936}, and η₁. The parameters were sampled from a lognormal distribution with 95% of values between ½ and 2 folds of their respective mean values. In addition to, and independently from these five parameters, VC, VP, and CL were sampled based on estimated between subject variability (BSV).

The model was developed in Matlab Simbiology (version 2021b). Simulations were done on SCP cluster using Matlab (version 2019b).

### Results

Sensitivity analysis was performed to evaluate the relative impact of key parameters on tumoral receptor occupancy prediction. From this analysis, in the dose ranges that are most likely to achieve full receptor occupancy (750 and 1500 mg), the predicted tumor receptor occupancy of PD-1 and TIGIT are most sensitive to tumor partition coefficient (PC), which determines the amount of drug that can penetrate the tumor tissue (i.e., tumor penetration), and initial receptor concentration (RC) in the tumor microenvironment. They are also sensitive to the internalization rates and equilibrium rate constant.

In these four parameters, PC and RC are highly variable among the patient population, while internalization rates and equilibrium rate constant are informed by internal and literature knowledge with some level of uncertainty. Without sufficient information to assign a probability distribution to PC and RC in patients, four groups were examined consisting of combinations using high and low values of PC and RC. For k_{int,PD}1 and k_{eq,AzD2936}, the groups sampled them from a distribution as described in section 2.5 to reflect the best knowledge while accounting for uncertainty in the values.

Virtual patients were divided into 4 subgroups, “high PC, low RC,” “high PC, high RC,” “low PC, low RC,” and “low PC, high RC” when predicting the receptor occupancy, based on their tumor penetration (PC=5% or 50%) and tumor RC (3 nM or 30 nM for PD-1; 4 nM or 40 nM for TIGIT, which can be calculated using RA₀,VT=1 or 10 nM, ψBA=1, ψCA=2, ψDA=3). High/low tumor PD-1/TIGIT receptor concentrations were calculated based on the assumption that receptor number per cell is 100,000, T cell area density in tumor ranges from 400 to 4,000 cell/mm², and that the ratio between PD-1+TIGIT+/PD-1+TIGIT−/PD-1-TIGIT+ cells remain the same between peripheral and tumor. 50% in high PC group was supported by experimental investigations using radiolabeled mAbs that measured tumor uptake. Since those data do not have sufficient granularity to quantitatively describe the spatial heterogeneity of mAb concentrations within a tumor lesion, a conservative estimate was applied to approximate the scenario in low PC group and resulted in ˜5% tumor concentration relative to blood (T. R. Li et al., Clinical Pharmacology & Therapeutics, 110(1):200-209, 2021). High/low tumor PD-1/TIGIT receptor concentrations were calculated based on the assumption that receptor number per cell is 100,000, T cell area density in tumor ranges from 500 to 5,000 cell/mm², and that the ratio between PD-1⁺TIGIT⁺/PD-1⁺TIGIT⁻/PD-1⁻-TIGIT⁺cells remain the same between peripheral and tumor.

The model predicts ≥9000 receptor occupancy for both PD-1 and TIGIT in blood throughout the dosing interval in ≥900% of patients starting at a dose of 70 mg regardless of patient subgroups. The predicted receptor occupancy of PD-1 and TIGIT in the tumor microenvironment, following IV administration of AZD2936 at 70,210,750, and 1500 mg Q3W are shown in FIG. 1 and Table 2. Doses of 70 and 210 mg Q3W are likely to show sub-optimal receptor occupancy (particularly PD-1) in patients with low tumor partition coefficient (i.e., low drug penetration into the tumor). The dose level of 750 mg Q3W is predicted to achieve intra-tumoral receptor occupancy of ≥90% in most patients across a broad spectrum of conditions. The dose level of 1500 mg Q3W is predicted to achieve better receptor occupancy than 750 mg Q3W, especially in the “low PC, high RC” group.

TABLE 2
Receptor Modeling Occupancy Results

| | | | | |
|---|---|---|---|---|
| | A1 | A2 | A3 | A4 |
| | 70 | 210 | 750 | 1500 |
| AZD2936 Dose | mg | mg | mg | mg |

High PC, low RC

| | | | | |
|---|---|---|---|---|
| PD-1 % RO >90% | ≥90% | ≥90% | ≥90% | ≥90% |
| TIGIT % RO >90% | ≥90% | ≥90% | ≥90% | ≥90% |

High PC, high RC

| | | | | |
|---|---|---|---|---|
| PD-1 % RO >90% | 74.2% | ≥90% | ≥90% | ≥90% |
| TIGIT % RO >90% | ≥90% | ≥90% | ≥90% | ≥90% |

Low PC, low RC

| | | | | |
|---|---|---|---|---|
| PD-1 % RO >90% | <1% | 49.5% | ≥90% | ≥90% |
| TIGIT % RO >90% | 86.6% | ≥90% | ≥90% | ≥90% |

Low PC, high RC

| | | | | |
|---|---|---|---|---|
| PD-1 % RO >90% | <1% | 9.7% | 78.5% | ≥90% |
| TIGIT % RO >90% | 8.2% | 54.1% | ≥90% | ≥90% |

The predicted RO for PD-1 in the tumor from different dose level of AZD2936 was compared with other anti-PD-1 drugs (i.e., nivolumab and pembrolizumab). The comparison was performed under the same four scenarios of PC/RC combinations. Binding affinity and population PK parameters for nivolumab and pembrolizumab were obtained from the literature and regulatory reports listed below, while other systems parameters and variabilities kept the same as AZD2936 simulations.

Using 240 mg Q2W for nivolumab and 200 mg Q3W for pembrolizumab, the simulation shows that tumor PD-1 RO is maintained above 90% at trough concentration in all four groups of patients. With AZD2936, the dose of 210 mg Q3W was predicted to have sub-optimal receptor occupancy in patients with low tumor partition coefficient compared with nivolumab and pembrolizumab in the same group of virtual patients.

### SUMMARY

The receptor occupancies of TIGIT and PD1 in blood and tumor were predicted when virtual patients were treated with AZD2936. The conclusions of this analysis are:
The model predicts ≥90% receptor occupancy for both PD-1 and TIGIT in blood throughout the dosing interval in ≥90% of patients starting at a dose of 70 mg, regardless of patient subgroups.
Sensitivity analysis shows tumor RO is significantly affected by tumor penetration and receptor concentration, which are highly variable in different patients. Due to the lack of data on the probability distribution of these two parameters among patients, tumor RO under different dose levels in each subgroup was assessed.
Doses of 70 and 210 mg Q3W predict sub-optimal receptor occupancy of PD-1 in patients with low tumor penetration. The dose level of 750 mg Q3W is predicted to achieve intra-tumoral receptor occupancy of ≥90% in most patients across a broad spectrum of conditions. The dose level of 1500 mg Q3W is predicted to achieve better receptor occupancy than 750 mg Q3W, especially in patients with low tumor penetration and high receptor concentration.

### Example 2: Study Design and Definitions

Multiple solid tumor malignancies including NSCLC are considered to be sensitive to immune checkpoint blockade. However, subjects who initially respond to immune-oncology (IO) therapy may experience disease progression due to primary or secondary resistance mediated by multiple mechanisms such as T cell exhaustion, insufficient T cell priming, and abnormal antigen presentation, and TIGIT has been identified as a key inhibitor of antitumor responses that can hinder multiple steps of the cancer immunity cycle. In vitro and in vivo studies have demonstrated the ability of AZD2936 to inhibit TIGIT and PD-1 binding to their primary ligands (CD155 and PD-L1, respectively), thereby promoting enhanced immune-mediated antitumor responses compared with targeting PD-1 alone. Therefore, targeting both PD-1 and TIGIT with AZD2936 provides clinical benefit to subjects with advanced or metastatic NSCLC.

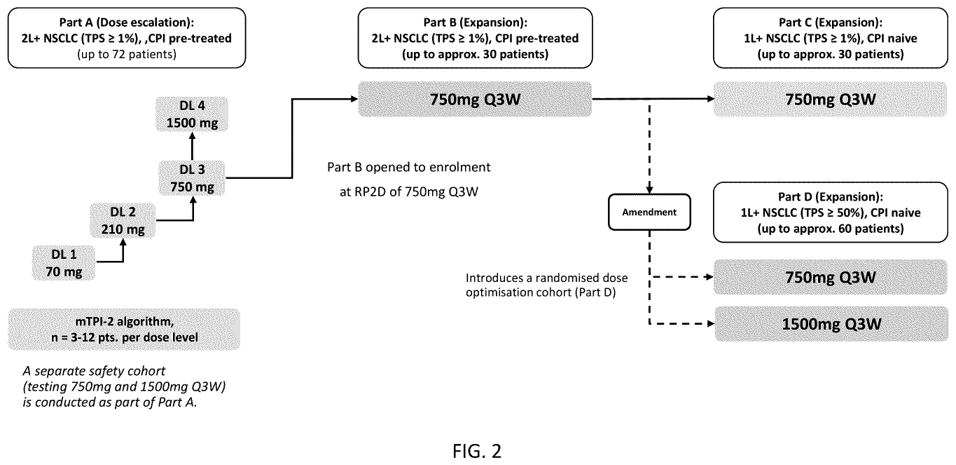

FIG. 2 shows a study flow diagram of the study outlined in the Examples below. The study is a first-time-in-human (FTIH), open-label, multicenter, multi-part, dose escalation and dose expansion study to evaluate the safety, PK, pharmacodynamics, and efficacy of AZD2936 in adult subjects with stage III unresectable or stage IV NSCLC.

Each of Part A to Part D of the study includes a screening period, which is from Day −28 to Day −1. The screening includes one visit with a medical professional, i.e., V1. During the screening period, the subjects are evaluated, which may include recording medical history, conducting a physical examination, testing blood and urine for safety, evaluating biomarkers, and sampling tumor tissue.

As shown in the schematic of FIG. 2, Part A to Part D is a sequential treatment study with 4 parts, with no masking.

Part A evaluates dose escalation of AZD2936 in second-line or greater (2L+) checkpoint inhibitor (CPI)-experienced subjects with stage III unresectable or stage IV NSCLC whose tumors express PD-L1 TPS ≥1%, and with primary or secondary resistance to a CPI-including regimen in order to determine a maximum tolerated dose (MTD), optimal biological dose (OBD), or maximum feasible dose (MFD), and a recommended Phase II dose (RP2D). Dose escalation follows the modified toxicity probability interval-2 (mTPI-2) algorithm consisting of up to 12 subjects per dose level. Intermediate dose levels are explored if warranted by emerging safety, pharmacokinetic (PK), pharmacodynamic, biomarker, and response data. Subjects are evaluated for dose-limiting toxicities (DLTs) during a 21-day DLT evaluation period.

Parts B-D are initiated once the MTD, OBD, or MFD and an RP2D are established in Part A (dose escalation). Parts B and C evaluate the safety, tolerability, and antitumor activity of AZD2936 at the RP2D determined during Part A (dose escalation) in 2 cohorts.

Part B (dose expansion) is performed on 2L+ CPI-experienced subjects with stage III unresectable or stage IV NSCLC whose tumors express PD-L1 TPS ≥1%. Subject criteria are the same as for Part A (dose escalation), with enrollment of both primary and secondary CPI-experienced subjects.

Part C (dose expansion) is performed on CPI-naïve subjects with stage IV NSCLC whose tumors express PD-L1 TPS ≥1%.

Part D incorporates a randomized design evaluating the safety, tolerability, and antitumor activity of AZD2936 at 750 mg and 1500 mg Q3W. Part D is designed to evaluate whether a dose-efficacy plateau is reached at 750 mg Q3W and to allow selection of the optimal dose for exploration in future studies. Part D runs in parallel to Part C where that cohort is already open, or Part D replaces Part C where that cohort is not already open. Part D (dose expansion) is performed on CPI-naïve subjects with stage IV NSCLC whose tumors express PD-L1 TPS ≥50%. Subjects are randomized 1:1 to either AZD2936 750 mg Q3W (RP2D) or AZD2936 1500 mg Q3W.

Definitions—The following terms are used in the study described in the Examples below.

Dose-limiting Toxicity (DLT). The DLTs will be evaluated during Part A (dose escalation). The DLT evaluation period will be 21 days from the first dose of AZD2936 on Cycle 1 Day 1. A DLT will be defined during Part A (dose escalation) as any ≥Grade 3 toxicity that occurs during the DLT evaluation period, with modifications or exceptions. Toxicity that is clearly attributable to the primary disease, other concomitant medications, disease-related processes under investigation, or another non-drug-related etiology (not related to treatment with study intervention) is excluded from this definition. All DLTs must be documented as AEs. All AEs will be graded according to NCI CTCAE v5.0.

Maximum Tolerated Dose (MTD). The MTD will be selected from all tried dose levels that have not been previously declared to be unsafe with a ‘DU’ (current dose is unacceptably toxic) decision according to the mTPI-2 algorithm. With this constraint, the MTD will be determined as the dose level with the DLT estimate closest to the target toxicity level of 30%.

In the case of dose levels with estimated toxicity of equal distance (tied dose levels) from the target toxicity of 30%, the following approach will be used (Ji Y, et al., A modified toxicity probability interval method for dose-finding trials, Clin Trials, 2010, 7(6):653-63) among all tied dose levels the highest dose level with target toxicity ≤30% will be selected, unless all tied dose levels have estimated toxicity >30%, in which case the lowest dose level will be selected.

Efficacy Assessments. Tumor response will be assessed by RECIST v1.1 (Eisenhauer et al, 2009) according to the schedule (Q9W [±7 days] for 54 weeks then Q18W [±14 days] relative to the date of first dose [Cycle 1 Day 1] until disease progression or initiation of other anticancer therapy).

Tumor Evaluation. Tumor assessments will include physical examination and cross-sectional imaging using CT (preferred) or MRI scans. The CT scan of the chest, and CT or MRI scan of the abdomen, and pelvis will be performed at screening (with contrast, unless the subject has a documented intolerance). The preferred method of systemic disease assessment is CT with contrast; if CT with contrast is contraindicated, CT without contrast is preferred over MRI. The preferred method for brain imaging is MRI over CT (with contrast, unless the subject has a documented intolerance). The preferred method for bone imaging is bone scan. At screening, bone imaging is only applicable for subjects in whom there is a clinical suspicion of or confirmed bone metastases. Follow-up brain and bone imaging is required as clinically indicated if there are no metastases at baseline, and with every restaging scan if baseline brain or bone metastases are present. The same method should be used for all subsequent tumor assessments. Tumor assessments prior to baseline may also be requested, if available, provided these exams were performed within 6 months prior to the start of treatment. Circulating tumor deoxyribonucleic acid (ctDNA) and tumor antigen markers will be used, where available, as exploratory markers in the assessment of disease response.

Eastern Cooperative Oncology Group Performance Status. The ECOG performance status will be assessed at specified time points.

Adverse Events. An adverse event (AE) is the development of any untoward medical occurrence in a patient or clinical study subject administered a medicinal product and which does not necessarily have a causal relationship with this treatment. An AE can therefore be any unfavorable and unintended sign (e.g., an abnormal laboratory finding), symptom (for example nausea, chest pain), or disease temporally associated with the use of a medicinal product, whether or not considered related to the medicinal product.

The term AE is used to include both serious and non-serious AEs and can include a deterioration of a pre-existing medical occurrence. An AE may occur at any time, including run-in or washout periods, even if no Study treatment has been administered.

Serious Adverse Events. A serious adverse event (SAE) is an AE occurring during any study phase (i.e., run-in, treatment, washout, follow-up), that fulfils one or more of the following criteria: (1) Results in death; (2) Is immediately life-threatening; (3) Requires subject hospitalization or prolongation of existing hospitalization; (4) Results in persistent or significant disability or incapacity; (5) Is a congenital anomaly or birth defect; (6) Is an important medical event that may jeopardize the subject or may require medical treatment to prevent one of the outcomes listed above.

Disease Progression. Disease progression can be considered as a worsening of a subject's condition attributable to the disease for which the investigational product is being studied. It may be an increase in the severity of the disease under study and/or increases in the symptoms of the disease. The development of new or progression of existing metastasis to the primary cancer under study should be considered as disease progression and not an AE. Events that are unequivocally due to disease progression should not be reported as AEs during the study.

New Cancers. The development of a new cancer should be regarded as an SAE. New primary cancers are those that are not the primary reason for the administration of the study intervention and have been identified after the subject's inclusion in this study. They do not include metastases of the original cancer.

### Example 3: Formulation and Administration of AZD2936

This example shows the formulation and administration of AZD2936. AZD2936 is supplied in Part A-Part D as either a lyophilized or liquid product.

Lyophilized AZD2936 is supplied in glass vials as a sterile, white to off-white, lyophilized product at a nominal fill volume of 5.0 mL for infusion after reconstitution. Each vial contains 250 mg (nominal) of active novel agent. Following reconstitution with 5.0 mL sterile water for injection (sWFI), each vial contains 50 mg/mL AZD2936. The reconstituted solution should appear clear to opalescent, colorless to slightly yellow, and free from visible particles. Following reconstitution with sWFI, AZD2936 is further diluted in 0.9% (w/v) saline.

Liquid AZD2936 is supplied in glass vials as a sterile solution at a nominal fill volume of 15.0 mL for infusion. Each vial contains 750 mg (nominal) of active novel agent.

The AZD2936 product vials are stored so as to prevent exposure to light. Investigational product vials are stored at 2° C. to 8° C. (36° F. to 46° F.) until ready for use.

AZD2936 is administered by IV infusion.

The dose of AZD2936 for administration is prepared using aseptic technique. Total time from needle puncture of the AZD2936 vial to the start of administration should not exceed 24 hours at 2° C. to 8° C. (36° F. to 46° F.) or should not exceed 4 hours at room temperature up to 25° C. (77° F.). Standard infusion time for AZD2936 is over 1 hour (up to a total of 90 minutes); however, if there are interruptions during infusion, the total allowed infusion time should not exceed 4 hours at room temperature. No other drugs are co-administered through the same infusion line. If either preparation time or infusion time exceeds the time limits, a new dose is prepared from new vials. AZD2936 does not contain preservatives.

Doses administered using IV bags are prepared as follows: doses of AZD2936 between 70 mg and 1500 mg are administered using an IV bag containing 0.9% (w/v) saline, with a final AZD2936 concentration ranging from 1 to 24 mg/mL and delivered through an IV administration set with a 0.2- or 0.22-μm filter. The required volume of AZD2936 (in mL) added to the IV bag for each dose level is calculated as follows:

$$Drug Product Volume ( mL ) = \frac{dose level ( mg )}{50 mg / ml}$$

where 50 mg/mL is the nominal AZD2936 drug product concentration.

### Example 4: Initial Risk/Benefit Assessment

Studies are conducted to assess risk of AD2936 administration and provide appropriate AZD2936 dose for Part A to Part D.

First, a no-observed-adverse-effect-level (NOAEL)-based FTIH starting dose was identified as 70 mg for a human based on a Good Laboratory Practice cynomolgus monkey toxicology study.

An initial risk assessment was then conducted based on the identified starting dose of 70 mg with dose escalation. A total of 64 2L+ subjects with NSCLC received AZD2936 therapy across 5 dose cohorts: Cohort A1, 70 mg (4 subjects), Cohort A2, 210 mg (11 subjects), Cohort A3, 750 mg (13 subjects), Cohort A4, 1500 mg (7 subjects), and Cohort B, 750 mg (29 subjects).

The results from this initial assessment show that AZD2936 was well tolerated across all the doses evaluated (up to and including 1500 mg Q3W). The safety profile of AZD2936 is similar across dose levels and no DLTs has been reported up to and including the 1500 mg Q3W regimen and therefore the MTD has not yet been reached during dose escalation. Additional safety assessments have been performed and are described below.

Preliminary signals of efficacy were observed in initial cohorts of 46 2L+ subjects with NSCLC, with 2 subjects achieving a partial response (one subject [9.1%] in Cohort A2 and one subject [8.3%] in Cohort A3). A total of 16 subjects achieved stable disease at ≥8 weeks; of these, 9 subjects (75%) were from Cohort A3 and treated with AZD2936 750 mg Q3W.

Preliminary PK data were evaluated using noncompartmental analysis in 32 subjects who received AZD2936 monotherapy at 70,210,750, and 1500 mg Q3W, in the dose escalation part of this study. AZD2936 systemic exposure over 70 to 1500 mg increases in a near dose-proportional manner. There was no target-mediated drug disposition observed at 70,210,750, and 1500 mg Q3W dose levels. Following a single dose IV administration, AZD2936 levels declined in a bi-phasic manner with a mean terminal elimination half-life (ti/2) of about 8 to 9 days. The steady state is achieved at approximately cycle 3, with minimal accumulation of AZD2936 observed following repeated dosing, with mean accumulation ratio ranging from 1.14 to 1.3 for Cₘₐₓ.

Based on the clinical samples analyzed in this study, AZD2936 achieves ≥90% PD-1 and TIGIT RO on peripheral T cells in all dose levels up to and including 1500 mg and PK/PD modelling predicted doses ≥750 mg to achieve optimal intra-tumoral PD-1 and TIGIT RO (≥90%).

### Example 5: Study Subjects

This Example provides criteria for selecting subjects for the Part A to Part D study.

Inclusion criteria for Part A-Part D:
1. Must be 18 years of age or over when enrolled in the study.
2. Histologically or cytologically documented squamous/non-squamous NSCLC not amenable to curative surgery or radiation as defined below (according to the 8th Edition of the International Association for the Study of Lung Cancer Staging Manual in Thoracic Oncology):
   Part A and Part B: stage III unresectable and stage IV squamous/non-squamous NSCLC.
   Part C and Part D: stage IV squamous/non-squamous NSCLC only.
3. Documented PD-L1 by PD-L1 IHC meeting the criteria below:
   Part A: PD-L1 TPS ≥1% per local report.
   Part B: PD-L1 TPS ≥1% per local report.
   Part C: PD-L1 TPS ≥1% per local report.
   Part D: PD-L1 TPS ≥50% per local report.
   Part C: must meet one of the definitions below:
      (i) No prior treatment for NSCLC, eligible for CPI-monotherapy because of high PD-L1 expression (TPS ≥50%), and not in need of rapid disease control via chemotherapy-containing regimen, or
      (ii) No prior treatment for NSCLC and not eligible for or not consenting to a platinum-based chemotherapy or combination regimen, or
      (iii) Prior treatment for NSCLC with one regimen consisting of chemotherapy only.
   Part D: must meet one of the definitions below:
      (i) No prior treatment for NSCLC, and not in need of rapid disease control via chemotherapy-containing regimen, or
      (ii) Prior treatment for NSCLC with one regimen consisting of chemotherapy only.
5. Body mass index ≥17.
6. Eastern Cooperative Oncology Group (ECOG) performance status of 0 or 1 at enrollment.
7. Predicted life expectancy of ≥12 weeks.
8. Must have at least one measurable lesion according to RECIST v1.1. (a) For subjects who undergo biopsies at screening and/or on treatment, the biopsied lesion must be distinct from any lesion used in the RECIST v1.1 evaluation.
9. Adequate organ and bone marrow function measured within 28 days prior to first dose.

The details of the criteria are shown in Table 3.

TABLE 3
Criteria for Adequate Organ and Marrow Function

| Type | Parameter | Value |
|---|---|---|
| Hematological | Hemoglobin | ≥9.0 g/dL (5.59 mmol/L) |
| | | with no blood transfusions |
| | | (packed red blood cells) within |
| | | 28 days prior to first dose |
| | Absolute neutrophil | ≥1.5 × 10⁹/L |
| | count | (1,500 per mm³) |
| | Platelet count | ≥100 × 10⁹/L (100,000 per mm³) |
| | | with no platelet transfusions |
| | | within 28 days prior to first dose |
| Hepatic | Total bilirubin | ≤1.5/ULN in the absence |
| | | of Gilbert's syndrome |
| | | ≤3 × ULN if the participant |
| | | has Gilbert's syndrome |
| | Alanine transaminase | ≤3 × ULN |
| | and aspartate | ≤5 × ULN in the case |
| | transaminase | of liver metastasis |
| Renal | Calculated creatinine | ≥45 mL/minute |
| | clearance by modified | |
| | Cockeroft Gault | |
| | (Rostoker et al, 2007) | |

ULN = upper limit normal.

### Example 6: Part A Dose Escalation

This Example describes the Part A dose escalation study to determine AZD2936 dosing for Part B and Part C.

The primary objectives of Part A dose escalation include assessing safety and tolerability, characterizing the DLTs, and determining the MTD, OBD, or MFD, and RP2D of AZD2936 in 2L+ CPI-experienced subjects with stage III/IV unresectable NSCLC with PD-L1 (TPS ≥1% using a PD-L1 immunohistochemistry (IHC) assay). The endpoints include the percentage of subjects with adverse events (AEs) and immune-mediated AEs (imAEs), serious AEs (SAEs), DLTs, vital signs, and abnormal laboratory parameters; and the rate of AZD2936 discontinuation due to toxicity.

The secondary objectives of the Part A dose escalation include determining the preliminary anti-tumor activity of AZD2936 in 2L+ CPI-experienced subjects with stage III/IV unresectable NSCLC with PD-L1 TPS ≥1%. The endpoints include, according to RECIST v1.1: ORR, disease control rate (DCR), duration of response (DoR), and durable response rate (DRR).

The secondary objectives of the Part A dose escalation also include evaluating the target engagement of AZD2936 in peripheral blood. The endpoints include measuring the receptor occupancy (RO) of TIGIT and PD-1 on peripheral blood T cells.

For all of Part A-Part D, the secondary objectives include assessing the pharmacokinetic (PK) profile compatibility of AZD2936 with Q3W dosing in 2L+ CPI-experienced and CPI-naïve subjects with stage III/IV unresectable NSCLC. The endpoints include serum concentrations and PK parameters (where applicable) of AZD2936; PK parameters to be evaluated include but not limited to maximum observed concentration (Cₘₐₓ), area under the concentration-time curve (AUC), clearance, and terminal elimination half-life (ti/2).

For all of Part A-Part D, the secondary objectives also include assessing the immunogenicity of AZD2936. The endpoints include Incidence of anti-drug antibodies (ADAs) against AZD2936 in serum.

Following an mTPI-2 dose escalation design for up to 6 dose levels, subjects were enrolled in Part A. Dose escalation to the next dose level is conducted using the mTPI-2 algorithm with a target DLT rate of 30% and an equivalence interval (25%, 35%). A minimum of 3 subjects had to complete the DLT-evaluation period before making an escalation decision. Dose escalation consists of at least 3 planned dose levels of AZD2936. Intermediate dose levels may be explored if warranted by emerging safety, PK, pharmacodynamics, biomarker, and efficacy data. Dose escalation and de-escalation recommendations followed the mTPI-2 algorithm. The mTPI-2 algorithm employs a simple beta-binomial Bayesian model. The prior distribution for all dose levels is Beta(1,1). The posterior density of the toxicity probability is divided into multiple intervals with equal length. These intervals are categorized as underdosing, proper dosing, and overdosing in terms of toxicity. The underdosing interval corresponds to a dose escalation, overdosing corresponds to a dose de-escalation, and proper dosing corresponds to staying at the current dose. Given an interval and a probability distribution, the unit probability mass of that interval is defined as the probability of the interval divided by the length of the interval. The design for the dose escalation phase of the study uses a target DLT rate of 30% and an equivalence interval (25%, 35%) for dose escalation/de-escalation decisions as well as MTD determination. A dose level will be considered unsafe, with no additional subjects enrolled at that dose level, if it has an estimated 95% or more probability of exceeding the target DLT rate of 30% (i.e., Probability [DLT >30% data]≥95%) with at least 3 subjects treated and evaluated at that dose level.

In Part A, four dose levels of 70 mg, 210 mg, 750 mg, and 1500 mg were used.

Following an initial screening period of up to 28 days, the Part A intervention period was scheduled. The Part A intervention period includes a maximum of 35 cycles, with each cycle 21 days, and AZD2936 is administered every three weeks (Q3W), i.e., once every cycle of 21 days.

Eligible subjects receive AZD2936 Q3W administered via intravenous (IV) infusion at the selected dose starting on Cycle 1 Day 1 for the maximum of 35 cycles. Subjects are treated with study intervention until disease progression, unacceptable toxicity, investigator's decision, completion of the maximum of 35 treatment cycles, or withdrawal of consent. All subjects are followed for survival until the end of the study. No dose reductions of AZD2936 are allowed at any time.

After the invention period, the subjects are further evaluated for disease progression, end of treatment condition, and follow-up condition.

The current data obtained during Part A shows that preliminary PK was near dose-proportional (70, 210, 750, and 1500 mg Q3W), with a limited impact of ADA. While in peripheral blood, all tested doses of AZD2936 achieved ≥90% RO for both PD-1 and TIGIT. Out of the doses tested in dose escalation, 750 mg Q3W was estimated to be the lowest dose to achieve ≥90% intra-tumoral receptor occupancy for both PD-1 and TIGIT in a large majority of subjects across a broad spectrum of conditions. Based on the PK/PD modelling analysis and emerging safety profile of AZD2936, 750 mg Q3W was identified as the RP2D for further evaluation in the expansion phase of this study (Parts B and C).

### Example 7: Part B Dose Expansion

The Part B study phase in this Example was conducted using a 750 mg dose of AZD2936 for studying dose expansion.

Parts B-D (dose expansion) are initiated after establishing the MTD, OBD, or MFD and the RP2D in Part A (dose escalation).

The primary objectives of the Part B dose expansion phase include assessing safety and tolerability of AZD2936 at the RP2D in 2L+ CPI-experienced subjects with stage III/IV unresectable NSCLC with PD-L1 TPS ≥1%. The endpoints include percentage of subjects with AEs and imAEs, SAEs, DLT-like events, vital signs, and abnormal laboratory parameters; and the rate of AZD2936 discontinuation due to toxicity.

The primary objects of the Part B dose expansion also include determining the preliminary anti-tumor activity of AZD2936 at the RP2D in 2L+ CPI-experienced subjects with stage III/IV unresectable NSCLC with PD-L1 (TPS ≥1% using PD-L1 IHC assay). The endpoints include Objective response rate (ORR) according to Response Evaluation Criteria in Solid Tumors (RECIST) v1.1.

The secondary objectives of the Part B dose expansion include determining the preliminary anti-tumor activity of AZD2936 at the RP2D in 2L+ CPI-experienced subjects with stage III/IV unresectable NSCLC with PD-L1 TPS ≥1%. The endpoints include According to RECIST v1.1: DCR, DoR, DRR, and progression-free survival (PFS).

The secondary objectives of Part B also include evaluating the target engagement of AZD2936 at the RP2D in peripheral blood. The endpoints include Measure the RO of TIGIT and PD-1 on peripheral blood T cells.

A sample size of at least 30 subjects was chosen to obtain a preliminary ORR with the standard error (SE) of not greater than 0.1. The following provides two-sided 80% confidence intervals (CIs) using the Clopper-Pearson method for a range of possible response rates out of 30 subjects:
3% ORR (1/30 responses); 80% CI [0.4%, 12%],
7% ORR (2/30 responses); 80% CI [2%, 17%],
10% ORR (3/30 responses); 80% CI [4%, 21%],
13% ORR (4/30 responses); 80% CI [6%, 25%],
20% ORR (6/30 responses); 80% CI [11%, 32%].

Based on a decision framework (Frewer P, et al., Decision-making in early clinical drug development, Pharm Stat, 2016, 15(3):255-63) with a target value (TV) of 15% in ORR, a conclusion of no evidence of targeted activity is reached if ≤1 objective RECIST response (confirmed complete response [CR] or partial response [PR]) is observed (observed ORR ≤3%). If the true response rate is 15%, the chance of observing ≤1 response in 30 evaluable subjects is ≤10%.

Based on the PK/PD modelling analysis and emerging safety profile of AZD2936, 750 mg Q3W was identified as the RP2D for further evaluation in the expansion phase of Part B and Part C.

After the screening period of up to 28 days, the Part B intervention period was scheduled. The Part B intervention period includes a maximum of 35 cycles, each cycle includes 21 days, and AZD2936 is administered every three weeks (Q3W), i.e., once every cycle of 21 days.

Particularly, eligible subjects receive AZD2936 Q3W administered via intravenous (IV) infusion at the RP2D (750 mg Q3W) starting on Cycle 1 Day 1 for the maximum of 35 cycles. Subjects are treated with study intervention until disease progression, unacceptable toxicity, investigator's decision, completion of the maximum of 35 treatment cycles, or withdrawal of consent.

After the intervention period, the subjects are further evaluated for disease progression, end of treatment condition, and follow-up condition.

### Example 8: Preliminary Part A and Part B Results

Eighty three subjects have been evaluated in the Part A and Part B portions of the study described in the Examples above.

Table 4 shows the demographics of the subjects evaluated in Part A and Part B. Table 5 shows the disease status of the 83 subjects evaluated in Part A and Part B. As shown in Table 5, most subjects present with metastatic disease and non-squamous histology.

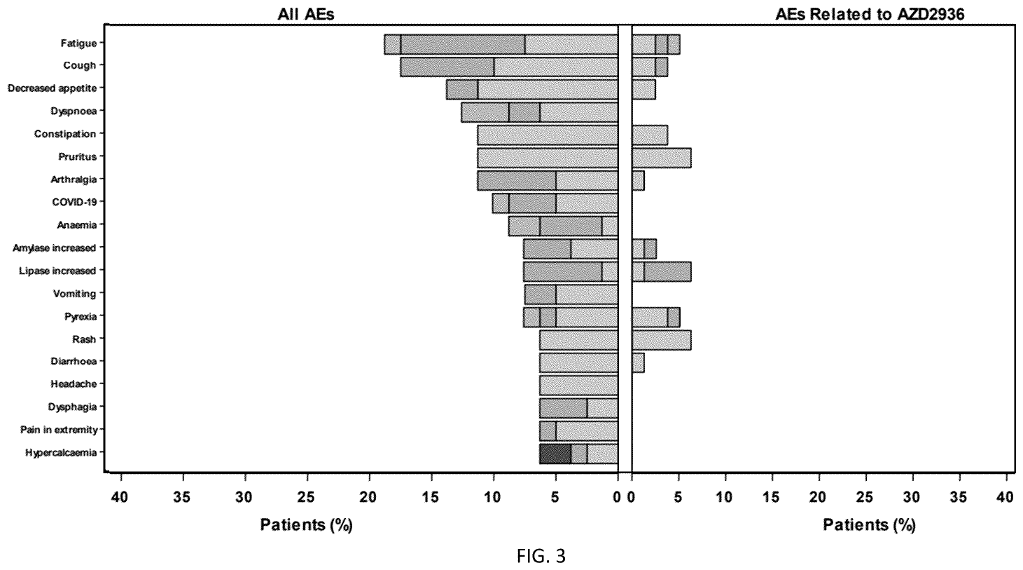

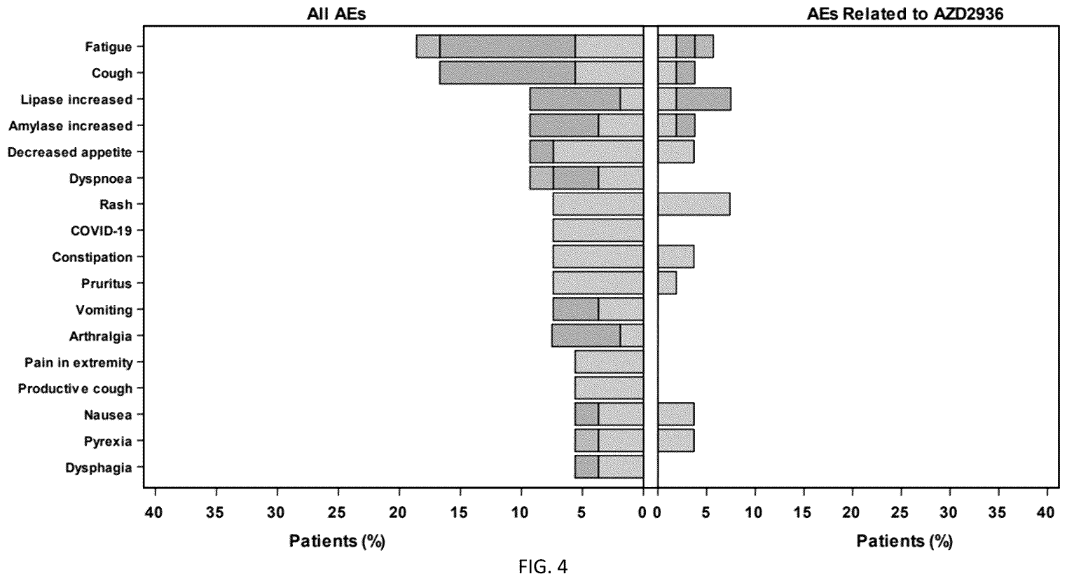

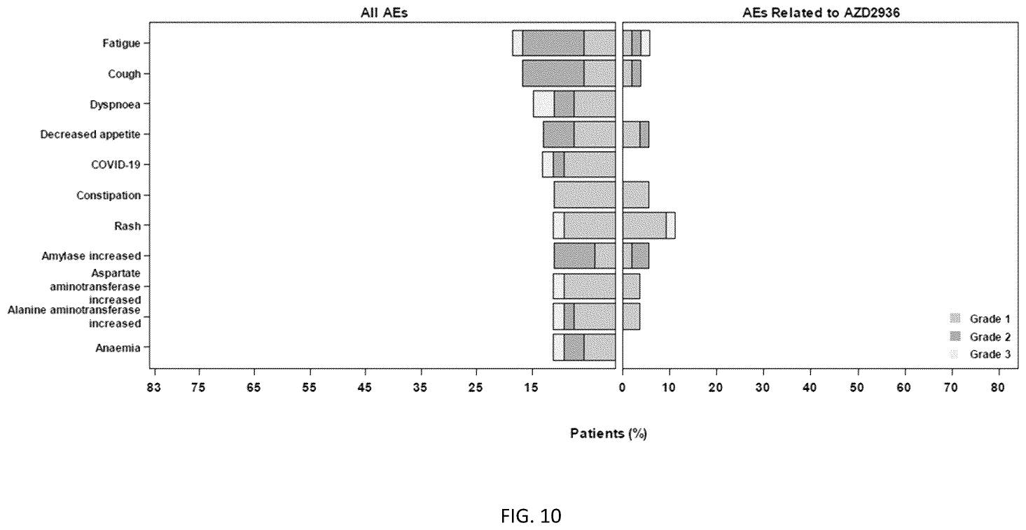

Adverse events occurring in greater than 5% of subjects in a preliminary analysis are plotted in FIG. 3. Adverse events related to AZD2936 are shown on the right side of the plot. Adverse events occurring in greater than 5% of subjects that were administered 750 mg AZD2936 in a preliminary analysis are plotted in FIG. 4. Adverse events occurring in greater than 10% of subjects dosed with AZD2936 750 mg Q3W are plotted in FIG. 10. Adverse events related to AZD2936 are shown on the right side of the plot. Nine serious adverse events (SAEs) were observed that were possibly related to AZD2936: one alanine aminotransferase increased (occurring in Part A4-JPN), one aspartate aminotransferase increased (occurring in Part A4-JPN), one colitis (occurring in Part A4), one diarrhoea (occurring in Part A4), one pneumonitis (occurring in Part B), one rash (occurring in Part B) and one suspected immune system disorder (occurring in Part A3); one case of acute hepatitis and one case of fatigue (both occurring in Part B).

TABLE 4
Demographics of Subjects Evaluated

| | Dose escalation | | | | | | | | All patients | |
|---|---|---|---|---|---|---|---|---|---|---|
| | | | | A3- | | | | Expansion | | A + B |
| | A1 | A2 | A3 | JPN¹ | A3+² | A4 | A4-JPN | B | A + B | all |
| | 70 mg | 210 mg | 750 mg | 750 mg | 750 mg | 1500 mg | 1500 mg | 750 mg | 750 mg | doses |
| | N = 4 | N = 11 | N = 12 | N = 6 | N = 4 | N = 11 | N = 3 | N = 32 | N = 54 | N = 83 |
| Age, median | 70.5 | 63.0 | 68.0 | 60.5 | 58 | 65.0 | 53.0 | 63.0 | 63.0 | 63.0 |
| Sex, n ( %) | 4 (100) | 3 | 5 | 1 | 1 | 7 (63.6) | 0 | 9 (28.1) | 16 | 30 |
| Female | 0 | (27.3) | (41.7) | (16.7) | (25.0) | 4 (36.4) | 3 (100) | 23 (71.9) | (29.6) | (36.1) |
| Male | | 8 | 7 | 5 | 3 | | | | 38 | 50 |
| | | (72.7) | (58.3) | (83.3) | (75.0) | | | | (70.4) | (63.9) |

TABLE 5
Disease Status of Subjects Evaluated

| | Dose escalation | | | | | | | | | |
|---|---|---|---|---|---|---|---|---|---|---|
| | | | | A3- | | | | Expansion | All patients | |
| | A1 | A2 | A3 | JPN | A3+ | A4 | A4-JPN | B | A + B | A + B |
| | 70 mg | 210 mg | 750 mg | 750 mg | 750 mg | 1500 mg | 1500 mg | 750 mg | 750 mg | all doses |
| | N = 4 | N = 11 | N = 12 | N = 6 | N = 4 | N = 11 | N = 3 | N = 32 | N = 54 | N = 83 |
| AJCC staging at | | | | | | | | | | |
| study entry, n ( %) | | | | | | | | | | |
| Stage I | 0 | 0 | 0 | 0 | 1 | 0 | 0 | 0 | 1 (1.9) | 1 (1.2) |
| Stage IA | 0 | 0 | 0 | 0 | (25.0) | 0 | 1 (33.3) | 0 | 0 | 1 (1.2) |
| Stage IB | 0 | 0 | 0 | 0 | 0 | 0 | 0 | 1 (1.2) | 1 (1.9) | 1 (1.2) |
| Stage IIB | 0 | 1 (9.1) | 0 | 0 | 0 | 1 (9.1) | 1 (33.3) | 1(1.2) | 1 (1.9) | 4 (4.8) |
| Stage IIIA | 1 (25.0) | 2 | 4 | 1 | 0 | 1 (9.1) | 0 | 4 (12.5) | 9 (16.7) | 13 |
| Stage IIIB | 1 (25.0) | (18.2) | (33.3) | (16.7) | 0 | 1 (9.1) | 0 | 3(9.4) | 5 (9.3) | (15.7) |
| Stage IIIC | 1 (25.0) | 2 | 1 (8.3) | 1 | 0 | 2 (18.2) | 0 | 2 (6.3) | 3 (5.6) | 9 (10.8) |
| Stage IV | 0 | (18.2) | 0 | (16.7) | 1 | 0 | 0 | 1 (3.1) | 2 (3.7) | 6 (7.2) |
| Stage IVA | 1 (25.0) | 0 | 0 | 0 | (25.0) | 1 (9.1) | 0 | 9 (28.1) | 15 | 3 (3.6) |
| Stage IVB | 0 | 1(9.1) | 3 | 0 | 1 | 5 (45.5) | 1 (33.3) | 11 (34.4) | (27.8) | 21 |
| | | 4 | (25.0) | 2 | (25.0) | | | | 17 | (25.3) |
| | | (36.4) | 4 | (33.3) | 1 | | | | (31.5) | 24 |
| | | 1 (9.1) | (33.3) | 2 | (25.0) | | | | | (28.9) |
| | | | | (33.3) | 0 | | | | | |
| Histology type, n ( %) | | | | | | | | | | |
| Squamous cell | 1 (25.0) | 5 | 4 | 0 | 1 | 2 (18.2) | 0 | 6 (18.8) | 11 | 19 |
| carcinoma | 0 | (45.5) | (33.3) | 0 | (25.0) | 0 | 0 | 0 | (20.4) | (22.9) |
| Small cell | 0 | 0 | 0 | 0 | 0 | 0 | 1 (33.3) | 0 | 0 | 0 |
| carcinoma | 3 (75.0) | 0 | 0 | 5 | 0 | 9 (81.8) | 2 (66.7) | 26 (81.3) | 0 | 1 (1.2) |
| Large cell carcinoma | 0 | 6 | 7 | (83.3) | 2 | 0 | 0 | 0 | 40 | 60 |
| Adenocarcinoma | 0 | (54.5) | (58.3) | 0 | (50.0) | 0 | 0 | 0 | (74.1) | (72.3) |
| Adenosquamous | 0 | 0 | 0 | 1 | 0 | 0 | 0 | 0 | 0 | 0 |
| Other | | 0 | 1 (8.3) | (16.7) | 1 | | | | 3 (5.6) | 3 (3.6) |
| Missing | | 0 | 0 | 0 | (25.0) | | | | 0 | 0 |
| | | | | | 0 | | | | | |

AJCC = American Joint Committee on Cancer

TABLE 6
Initial Efficacy Results for Studies

| | Dose escalation | | | | | | | | All patients | |
|---|---|---|---|---|---|---|---|---|---|---|
| | | | | A3- | | | A4- | Expansion | | A + B |
| | A1 | A2 | A3 | JPN | A3+ | A4 | JPN | B | A + B | all |
| Interim response- | 70 mg | 210 mg | 750 mg | 750 mg | 750 mg | 1500 mg | 1500 mg | 750 mg | 750 mg | doses |
| evaluable subjects⁺ | N = 4 | N = 11 | N = 12 | N = 6 | N = 4 | N = 11 | N = 3 | N = 32 | N = 54 | N = 83 |
| Overall response rate, | 1 (25.0) | 1 (9.1) | 1 (8.3) | 1 | 0 | 0 | 0 | 1 (3.1) | 3 (5.6) | 5 (6.0) |
| confirmed, % | | | | (16.7) | | | | | | |
| Complete response (CR), | 0 | 0 | 0 | 0 | 0 | 0 | 0 | 0 | 0 | 0 |
| confirmed, n ( %) | | | | | | | | | | |
| Partial response (PR), | 1 (25.0) | 1 (9.1) | 1 (8.3) | 1 (16.7) | 0 | 0 | 0 | 1 (3.1) | 3 (5.6) | 5 (6.0) |
| confirmed, n ( %) | | | | | | | | | | |
| CR or PR, unconfirmed, | 0 | 0 | 1 (8.3) | 0 | 0 | 0 | 1 (33.3) | 0 | 2 (3.7) | 3 (3.6) |
| n ( %) | | | | | | | | | | |
| Stable disease (≥8 | 2 (50.0) | 1 (9.1) | 9 | 3 | 0 | 5 (45.5) | 1 (33.3) | 12 (37.5) | 24 | 33 |
| weeks), n (%) | | | (75.0) | (50.0) | | | | | (44.4) | (39.8) |
| Progression | 1 (25.0) | 9 | 2 | 2 | 3 | 5 (45.5) | 2 (66.7) | 19 (59.4) | 24 | 43 |
| | | (81.8) | (16.7) | (33.3) | (75.0) | | | | (44.4) | (51.8) |
| Not evaluable*, n ( %) | 0 | 0 | 0 | 0 | 1 | 1 (9.1) | 0 | 0 | 1 (1.9) | 2 (2.4) |
| | | | | | (25.0) | | | | | |
| Disease control rate | 75.0 | 18.2 | 83.3 | 66.7 | 0 | 45.5 | 33.3 | 40.6 | 50.0 | 45.8 |
| (DCR), % | | | | | | | | | | |

⁺All dosed subjects who have measurable disease at baseline and who have first dose at least 9 weeks prior to data extract (where 9 weeks is the protocolled time between scans).
*No disease assessment at the time of DCO

TABLE 7
Summary of Results

| | AZD2936 | AZD2936 |
|---|---|---|
| | (all doses) | (750 mg q3w) |
| Efficacy | N = 83 | N = 54 |
| Response (confirmed), n ( %) | | |
| Complete response (CR) | 0 | 0 |
| Partial response (PR) | 5 (6.0) | 3 (5.6) |
| Overall response rate, % | 6.0 | 5.6 |
| Non-response, n ( %) | | |
| Stable disease | 33 (39.8) | 24 (44.4) |
| Progression | 43 (51.8) | 24 (44.4) |
| Disease control rate (DCR), % | 45.8 | 50 |
| Progression-free survival, | 2.1 (2.0, 4.0) | 3.8 (2.0, 4.2) |
| month, median (95% CI) | | |
| 6-month progression-free survival, % | 30.5 | 31.5 |
| Safety | N = 83 | N = 54 |
| Any TEAEs, n ( %) | 75 (90.4) | 51 (94.4) |
| possibly related to study treatment | 45 (54.2) | 33 (61.1) |
| Any TEAE ≥Grade 3, n ( %) | 28 (33.7) | 17 (31.5) |
| possibly related to study treatment | 6 (7.2) | 4 (7.4) |
| Any AE leading to discontinuation, n ( %) | 3 (3.6) | 2 (2.4) |
| ECOG performance status, ECOG 0, | 26 (31.3)/ | 17 (31.5)/ |
| n ( %)/ECOG 1 n, ( %) | 57 (68.7) | 37 (68.5) |

As can be seen in Table 6, at 750 mg Q3W dosing, the overall response rate (ORR) was 5.6% with 24 subjects having stable disease for a DCR of 50.0%. The DCR difference at 750 mg between Part A and Part B is likely explained by a heterogeneous patient population with enrichment of patients with adverse disease characteristics in Part B.

Tables 6 and 7 show a summary of the initial results obtained in Part A and Part B in second-line or greater (2L+) checkpoint inhibitor (CPI)-experiences participants. 83 subjects with pre-treated NSCLC were treated as described above. AZD2936 continues to show a favorable safety profile, and this safety profile may evolve further with longer follow-up and with transitioning into CPI naïve subjects.

### Example 9: Part C Dose Expansion

This Example describes using the 750 mg dose of AZD2936 for a further dose expansion study.

The primary objectives of Part C include assessing safety and tolerability of AZD2936 at the RP2D in CPI-naïve subjects with stage IV NSCLC with PD-L1 (TPS ≥1% using PD-L1 IHC assay). The endpoints include the Percentage of subjects with AEs and imAEs, SAEs, DLT-like events, vital signs, and abnormal laboratory parameters; and the rate of AZD2936 discontinuation due to toxicity.

The primary objectives of Part C also include determining the preliminary anti-tumor activity of AZD2936 at the RP2D in CPI-naïve subjects with stage IV NSCLC with PD-L1 TPS ≥1%. The endpoints include ORR according to RECIST v1.1.

The secondary objectives of Part C include further characterizing the preliminary anti-tumor activity of AZD2936 at the RP2D in CPI-naïve subjects with stage IV NSCLC with PD-L1 TPS ≥1%. The endpoints include, according to RECIST v1.1: DCR, DoR, DRR, and PFS.

A sample size of at least 30 subjects would obtain a preliminary ORR with the SE of not greater than 0.1. The following provides two-sided 80% CIs using the Clopper-Pearson method for a range of possible response rates out of 30 subjects:
20% ORR (6/30 responses); 80% CI [11%, 32%],
30% ORR (9/30 responses); 80% CI [19%, 43%],
40% ORR (12/30 responses); 80% CI [28%, 53%],
47% ORR (14/30 responses); 80% CI [34%, 60%],
60% ORR (18/30 responses); 80% CI [47%, 72%].

Based on a decision framework (Frewer et al, 2016) with a TV of 40% in ORR, a conclusion of no evidence of targeted activity is reached if ≤8 objective RECIST responses (confirmed CR or PR) are observed (observed ORR ≤27%). If the true response rate is 40%, the chance of observing ≤8 responses in 30 evaluable subjects is ≤10%.

During the execution of Part C, the occurrence of toxicities which are not clearly attributable to the primary disease, other concomitant medications, disease-related processes under investigation, or another non-drug-related etiology (not related to treatment with study intervention) will trigger automatic stopping rules. In this scenario, enrollment of new subjects into the study will be immediately halted, until the Safety Review Committee assesses the benefit/risk of AZD2936, if one of the following conditions occur:
1. One Grade 5 related toxicity at any time during Part C execution;
2. Two Grade 4 related toxicities in two different subjects at any time during Part C execution;
3. The occurrence of ≥4 events in different subjects (i.e., ≥2 events occurring in the same subject will be counted only once) featured as serious adverse drug reaction (SADR) in the first 10 enrolled subjects or the occurrence of ≥7 SADRs in the first 20 subjects.

Subjects are enrolled in Part C. Following an initial screening period of up to 28 days, eligible subjects receive AZD2936 Q3W administered via intravenous (IV) infusion at the RP2D (750 mg Q3W) starting on Cycle 1 Day 1 for a maximum of 35 cycles. Subjects are treated with study intervention until disease progression, unacceptable toxicity, investigator's decision, completion of the maximum of 35 treatment cycles, or withdrawal of consent. All subjects are followed for survival until the end of the study. No dose reductions of AZD2936 are allowed at any time.

After the screening period, the Part C intervention period is scheduled. The Part C intervention period includes a maximum of 35 cycles, each cycle includes 21 days, and AZD2936 is administered every three weeks (Q3W), i.e., once every cycle of 21 days.

Particularly, eligible subjects receive AZD2936 Q3W administered via intravenous (IV) infusion at the RP2D (750 mg Q3W) starting on Cycle 1 Day 1 for the maximum of 35 cycles. Subjects are treated with study intervention until disease progression, unacceptable toxicity, investigator's decision, completion of the maximum of 35 treatment cycles, or withdrawal of consent. All subjects are followed for survival until the end of the study. No dose reductions of AZD2936 are allowed at any time.

After the invention period, the subjects are further evaluated for disease progression, end of treatment condition, and follow-up condition.

### Example 10: Part D Dose Expansion

This Example describes using the 750 mg dose (Part D1) and 1500 mg dose (Part D2) of AZD2936 for a dose expansion study.

The primary objectives of Part D include assessing the safety and tolerability of AZD2936 at 750 mg and 1500 mg every 3 weeks (Q3W) in CPI-naïve subjects with stage IV NSCLC with PD-L1 TPS ≥50%. The endpoints include the percentage of subjects at each dose level with AEs and imAEs, SAEs, DLT-like events, vital signs, and abnormal laboratory parameters; and the rate of AZD2936 discontinuation due to toxicity at each dose level.

The primary objectives of Part D also include assessing the preliminary anti-tumor activity of AZD2936 at 750 mg and 1500 mg Q3W in CPI-naïve subjects with stage IV NSCLC with PD-L1 TPS ≥50%. The endpoints include ORR at each dose level according to RECIST v1.1.

The secondary objectives of Part D include further characterizing the preliminary anti-tumor activity of AZD2936 at 750 mg and 1500 mg Q3W in CPI-naïve subjects with stage IV NSCLC with PD-L1 (TPS ≥50% using PD-L1 IHC assay). The endpoints include, at each dose level, according to RECIST v1.1: DCR, DoR, DRR, and PFS.

Subjects are enrolled for the primary analysis in Part D, with subjects randomized 1:1 to either AZD2936 750 mg Q3W (RP2D; n=30) or AZD2936 1500 mg Q3W (n=30). A sample size of at least 30 subjects at each dose level would obtain a preliminary ORR with the SE of not greater than 0.1. The following provides two-sided 80% Cis using the Clopper-Pearson method for a range of possible response rates out of 30 subjects:
20% ORR (6/30 responses); 80% CI [11%, 32%],
30% ORR (9/30 responses); 80% CI [19%, 43%],
40% ORR (12/30 responses); 80% CI [28%, 53%],
47% ORR (14/30 responses); 80% CI [34%, 60%],
60% ORR (18/30 responses); 80% CI [47%, 72%].

Based on a decision framework (Frewer et al, 2016) with a TV of 45% in ORR, a conclusion of no evidence of targeted activity would be reached if ≤9 objective RECIST responses (confirmed CR or PR) are observed (observed ORR ≤30%). If the true response rate is 45%, the chance of observing ≤9 responses in 30 evaluable subjects is ≤10%.

During the execution of Part D, the occurrence of toxicities which are not clearly attributable to the primary disease, other concomitant medications, disease-related processes under investigation, or another non-drug-related etiology (not related to treatment with study intervention) will trigger automatic stopping rules (thereafter defined as stopping rules). In this scenario, enrollment of new subjects into the study will be immediately halted, until the Safety Review Committee assesses the benefit/risk of AZD2936, if one of the following conditions occur:
1. One Grade 5 related toxicity at any time during Part D execution;
2. Two Grade 4 related toxicities in two different subjects at the same dose level at any time during Part D execution;
3. The occurrence of ≥4 events in different subjects (i.e., ≥2 events occurring in the same subject will be counted only once) featured as SADR in the first 10 enrolled subjects or the occurrence of ≥7 SADRs in the first 20 subjects at each dose level.

After the screening period, Part D intervention period is scheduled. Part D intervention period includes a maximum of 35 cycles, each cycle includes 21 days, and AZD2936 is administered every three weeks (Q3W), i.e., once every cycle of 21 days.

Particularly, eligible subjects will receive AZD2936 Q3W administered via intravenous (IV) infusion at a dose of 750 mg or 1500 mg Q3W (1:1 randomization) starting on Cycle 1 Day 1 for the maximum of 35 cycles. Subjects are treated with study intervention until disease progression, unacceptable toxicity, investigator's decision, completion of the maximum of 35 treatment cycles, or withdrawal of consent. All subjects are followed for survival until the end of the study. After the intervention period, the subjects are further evaluated for disease progression, end of treatment condition, and follow-up condition.

As described in Part A above, 750 mg Q3W of AZD2936 was identified as the RP2D for further evaluation in the expansion phase of Part D. Further, a higher dose of 1500 mg Q3W of AZD2936 is evaluated alongside the PR2D of 750 mg Q3W. The dose of 1500 mg was declared safe and tolerably by SRC. The dose of 1500 mg was chosen based on emerging data showing a similar peripheral blood target engagement of ≥90% RO at 750 mg at preliminary analysis and predicted to achieve ≥90% intra-tumoral RO for both PD-1 and TIGIT. In contrast, as shown in Example 1 above, a lower dose of 210 mg is predicted to achieve suboptimal intra-tumoral RO compared to 750 mg and 1500 mg. In exploring the 1500 mg dose level, Part D evaluates whether a dose-efficacy plateau is reached at 750 mg Q3W and allow selection of the optimal dose for exploration in future studies.

In Part D, potential bias is reduced by the following steps: all subjects are assigned to randomized study intervention using an Interactive Response Technology (IRT)/Randomization and Trial Supply Management (RTSM).

### Example 11: Expanded Results of Part A and Part B Dose Escalation Study

The Part A dose escalation and Part B dose expansion study of AZD2936 described above was continued. Inclusion criteria for subjects in Part A and Part B was as provided in Example 5 above, and includes: (i) must have received both a prior CPI and prior platinum-based chemotherapy (combination or separate lines of treatment); (ii) must have confirmed progression during treatment with a CPI-containing regimen; (iii) must have no epidermal growth factor receptor (EGFR) mutation, anaplastic lymphoma kinase (ALK) fusion or other known genomic alteration with a targeted therapy approved in first line; and (iv) must have no discontinuation of prior CPI treatment due to toxicity. Abbreviations are as defined in Example 2 above. 83 subjects were enrolled and treated: 51 in Part A and 32 in Part B. The subjects had Stage III unresectable or Stage IV NSCLC previously treated with a CPI, with PD-L1 TPS ≥1%.

AZD2936 was administered intravenously once every three weeks (Q3W). The demographics of the subjects are provided in Table 8.

TABLE 8
Baseline Demographics and Disease Characteristics

| | Dose escalation | Dose expansion |
|---|---|---|
| | (Part A) | (Part B) |
| | 70-1500 mg Q3W | 750 mg Q3W |
| | (N = 51) | (N = 32) |
| Median age (range), | 65.0 (39-83) | 63.0 (41-85) |
| years | | |
| Male/Female, n (%) | 30 (58.8)/21 (41.2) | 23 (71.9)/9 (28.1) |
| White/Asian/other, | 27 (52.9)/22 (43.1)/2 (3.9) | 20 (62.5)/12 (37.5)/0 |
| n (%) | | |
| ECOG PS 0/1, n (%) | 20 (39.2)/31 (60.8) | 6 (18.8)/26 (81.3) |
| Histology, n (%) | | |
| Adenocarcinoma | 34 (66.7) | 26 (81.3) |
| Squamous | 13 (25.5) | 6 (18.8) |
| PD-L1 TPS 1-49%/ | 28 (54.9)/23 (45.1) | 16 (50.0)/16 (50.0) |
| ≥50%, n (%) | | |
| Current or former | 41 (80.4)/10 (19.6) | 26 (81.3)/6 (18.8) |
| smoker/never | | |
| smoker, n (%) | | |
| Primary/secondary | 19 (37.3)/32 (62.7) | 12 (37.5)/20 (62.5) |
| resistance* to prior | | |
| CPI treatment, n (%) | | |
| Liver metastasis/brain | 4 (7.8)/5 (9.8) | 6 (18.8)/13 (40.6) |
| metastases, n(%) | | |

Primary resistance is defined as resistance with exposure to CPI therapy <6 months, and secondary resistance is defined as resistance with exposure to CPI therapy ≥6 months. ECOG PS means Eastern Cooperative Oncology Group Performance Status. As can be seen in Table 8, most subjects had acquired resistance to prior CPI treatment.

The median duration of treatment was 14 weeks (range: 1.7-95.6) for Part A and 10.7 weeks (range: 5.9-60.7) for Part B as of the time of data collection. There were no dose-limiting toxicities (DLTs) observed during dose escalation (Part A). The RP2D) was 750 mg based on data from Part A and supported by modelling analysis of predicted intratumoural receptor occupancy. A safety summary of the study is provided in Table 9. Treatment-emergent adverse events (TEAEs) occurred in 90.4% o of subjects and were grade 3 or higher in 33.700 for the 70-1500 mg dose, and occurred in 94.400 of subjects and were grade 3 or higher in 31.5% o for the 750 mg dose. Treatment-related adverse events (TRAEs) occurred in 53.0% of subjects and were grade 3 or higher in 8.400 for the 70-1500 mg range, and occurred in 59.300 of subjects and were grade 3 or higher in 9.300 for the 750 mg range.

TABLE 9
Safety Summary

| | | | | | | Dose | | |
|---|---|---|---|---|---|---|---|---|
| | Dose Escalation | | | | | Expansion | Total | |
| | (Part A) | | | | | (Part B) | (Parts A and B) | |
| | 70 mg | 210 mg | 750 mg | 1500 mg | 70-1500 mg | 750 mg | 750 mg | 70-1500 mg |
| Event, n (%) | (n = 4) | (n = 11) | (n = 22) | (n = 14) | (n = 51) | (n = 32) | (n = 54) | (n = 83) |
| Any TEAE | 3 (75.0) | 9 (81.8) | 20 (90.9) | 12 (85.7) | 44 (86.3) | 31 (96.9) | 51 (94.4) | 75 (90.4) |
| Any TRAE* | 1 (25.0) | 4 (36.4) | 14 (63.6) | 7 (50.0) | 26 (51.0) | 18 (56.3) | 32 (59.3) | 44 (53.0) |
| Any Grade ≥3 | 1 (25.0) | 3 (27.3) | 6 (27.3) | 7 (50.0) | 17 (33.3) | 11 (34.4) | 17 (31.5) | 28 (33.7) |
| TEAE | | | | | | | | |
| Any Grade ≥3 | 0 | 0 | 2 (9.1) | 2 (14.3) | 4 (7.8) | 3 (9.4) | 5 (9.3) | 7 (8.4) |
| TRAE* | | | | | | | | |
| Any treatment- | 1 (25.0) | 3 (27.3) | 7 (27.3) | 6 (42.9) | 17 (33.3) | 11 (34.4) | 18 (33.3) | 28 (33.7) |
| emergent SAE | | | | | | | | |
| Any treatment- | 0 | 0 | 1 (4.5) | 2 (14.3) | 3 (5.9) | 3 (9.4) | 4 (7.4) | 6 (7.2) |
| related SAE* | | | | | | | | |
| Any treatment- | 1 (25.0) | 0 | 1 (4.5) | 2 (14.3) | 4 (7.8) | 0 | 1 (1.9) | 4 (4.8) |
| emergent SAE | | | | | | | | |
| leading to death | | | | | | | | |
| Any treatment- | 0 | 0 | 0 | 0 | 0 | 0 | 0 | 0 |
| related SAE | | | | | | | | |
| leading to death* | | | | | | | | |
| Any TEAE | 0 | 0 | 1 (4.5) | 0 | 1 (2.0) | 2 (6.3) | 3 (5.6) | 3 (3.6) |
| leading to | | | | | | | | |
| discontinuation | | | | | | | | |
| Any imAE^{†} | 1 (25.0) | 2 (18.2) | 3 (13.6) | 3 (21.4) | 9 (17.6) | 5 (15.6) | 8 (14.8) | 14 16.9) |

*Possibly related to AZD2936 as assessed by the investigator.
^{†}Immune-mediated as assessed by the investigator. AE, adverse event; AESI, adverse event of special interest; imAE, immune-mediated adverse event; SAE, serious adverse event; TEAE, treatment-emergent adverse event; TRAE, treatment-related adverse event.

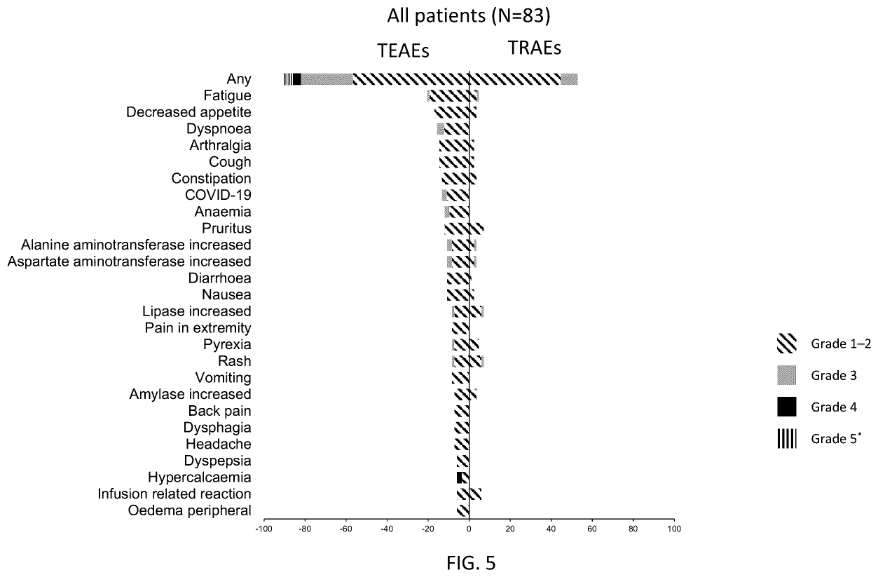

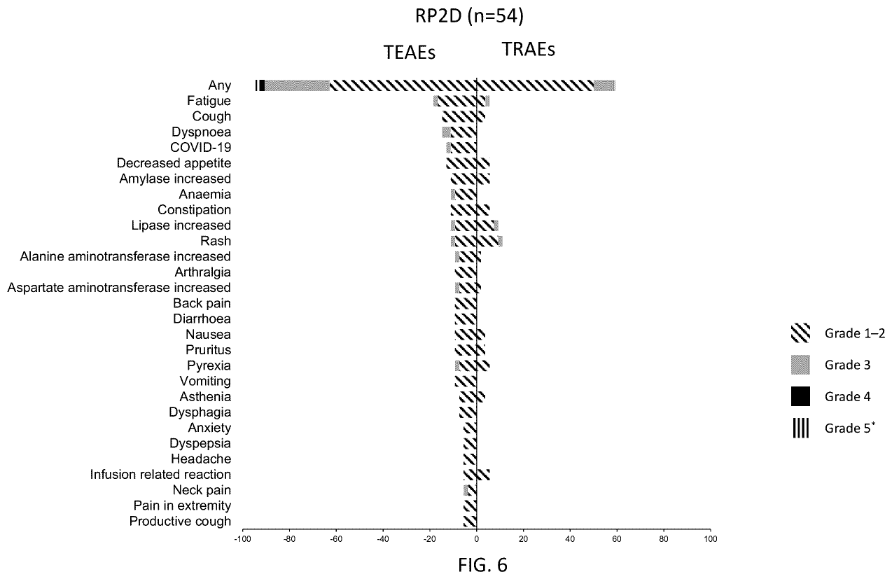

FIG. 5 summarizes the TEAEs and TRAEs occurring in >5% of the 83 subjects treated with AZD2936 (20-1,500 mg Q3W). FIG. 6 summarizes the TEAEs and TRAEs occurring in >5% of the subset ofD54 patients with AZD2936 treatment at the recommended phase 2 dose (750 mg Q3W). As shown by the plots in FIG. 5 and FIG. 6, there were no Grade 4 or 5 TRAEs. Thirteen (15.70) patients received steroids to treat TRAEs. Three (3.60) patients, all receiving 750 mg, discontinued AZD2936 due to AEs. These AEs were considered treatment-related in two patients, both of whom recovered with steroids: myocarditis (Grade 1) and acute hepatitis (Grade 3). Four (4.8%) patients had AEs leading to death, all in Part A: ileus (n=2), pneumonia (n=1), and respiratory failure (n=1). None were considered related to AZD2936.

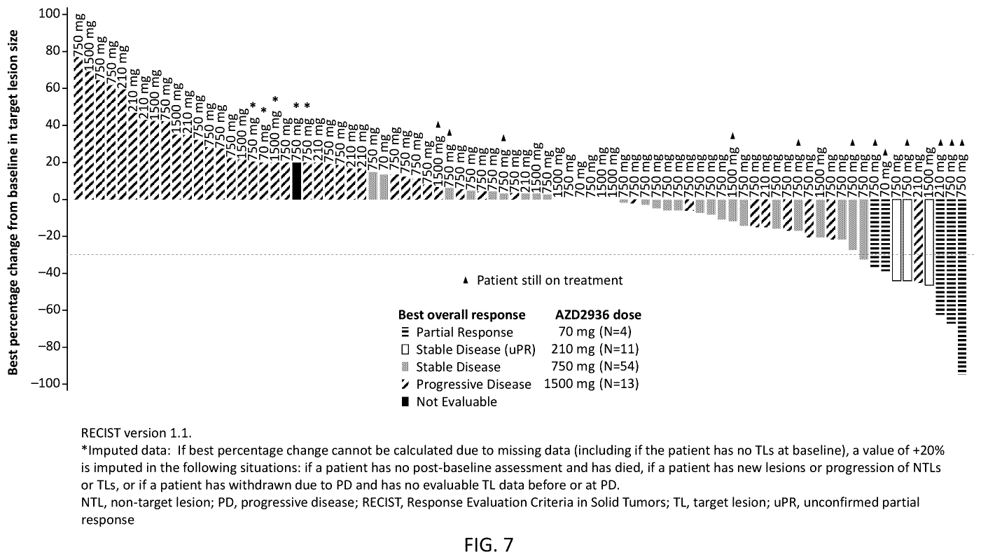

The efficacy of AZD2936 was analyzed by measuring change in target lesion size. FIG. 7 is a waterfall plot ofbest percentage change from baseline in target lesion size for all doses in 82 subjects who were analyzed. Table 10 shows a summary of antitumour activity in all 83 subjects. Overall, median PFS is 2.1 months and ORR is 6.0%.

TABLE 10
Antitumour activity in subjects with CPI-resistant NSCLC

| | Dose | Dose | | |
|---|---|---|---|---|
| | Escalation | Expansion | Total | |
| | (Part A) | (Part B) | (Parts A and B)) | |
| | 70-1500 mg | 750 mg | 750 mg | 70-1500 mg |
| | (n = 51) | (n = 32) | (n = 54) | (N = 83) |
| ORR, % (95% CI) | 7.8 (2.2, 18.9) | 3.1 (0.1, 16.2) | 5.6 (1.2, 15.4) | 6.0 (2.0, 13.5) |
| Complete response, n (%) | 0 | 0 | 0 | 0 |
| Partial response, n (%) | 4 (7.8) | 1 (3.1) | 3 (5.6) | 5 (6.0) |
| Median DoR (range), | 14.5 (4.8-14.5) | 4.1 (NC) | 14.5 (4.1-14.5) | 14.5 (4.1-14.5) |
| months* | | | | |
| Durable response at 6 | 3 (5.9) | 0 | 2 (3.7) | 3 (3.6) |
| months, n (%) | | | | |
| Stable disease, n (%) | 21 (41.2) | 12 (37.5) | 24 (44.4) | 33 (39.8) |
| Unconfirmed complete or | 2 (3.9) | 1 (3.1) | 2 (3.7) | 3 (3.6) |
| partial response, n (%) | | | | |
| Disease control rate, % | 31.4 (19.1-45.9) | 28.1 (13.8-46.8) | 31.5 (19.5-45.6) | 30.1 (20.5-41.2) |
| (95% CI)† | | | | |
| Median PFS (95% CI), | 3.1 (2.0, 4.1) | 2.1 (2.0, 5.2) | 3.8 (2.0, 4.2) | 2.1 (2.0, 4.0) |
| months | | | | |
| Median duration of follow- | 9.1 (0.4-21.3) | 10.1 (1.6-13.7) | 9.9 (0.8-17.9) | 9.7(0.4-21.3) |
| up (range), months | | | | |

*DoR is calculated based on the Kaplan-Meier technique;
†Disease control = complete response + partial response + stable disease at or after 182 days;
CI, confidence interval;
DoR, duration of response;
NC, not calculable;
ORR, objective response rate;
PFS, progression-free survival.

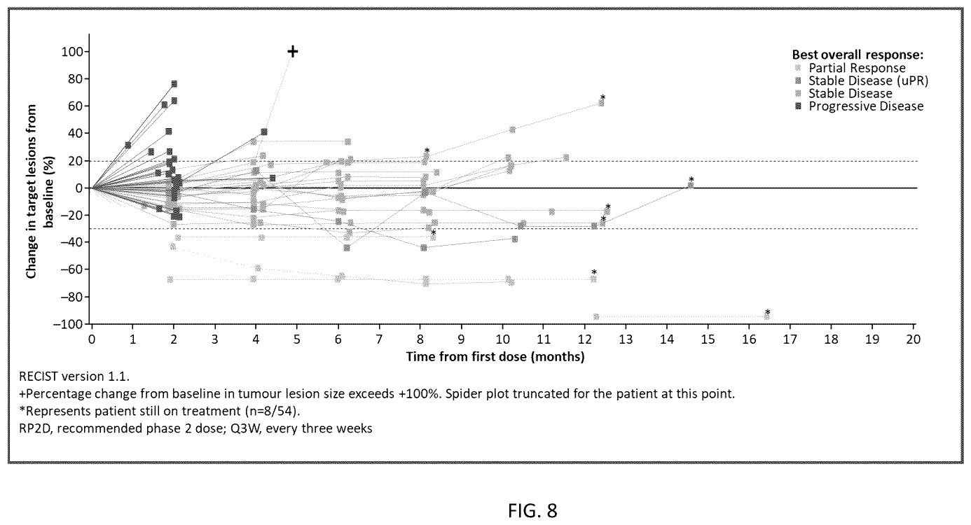

FIG. 8 shows percentage change from baseline in target lesion size over time in the subset of 54 subjects treated at the recommended phase 2 dose (750 mg Q3W, Parts A and B), as summarized in column 3 of Table 10. The median PFS is 3.8 months and ORR is 5.6%. Responses and stable disease were generally durable at this dose.

From the efficacy results as described above, twenty patients had stable disease lasting at least 6 months, most of whom (n=18) received doses of 750 mg or 1500 mg; they had PD-L1 TPS 1-49% (n=10) or ≥50% (n=10).

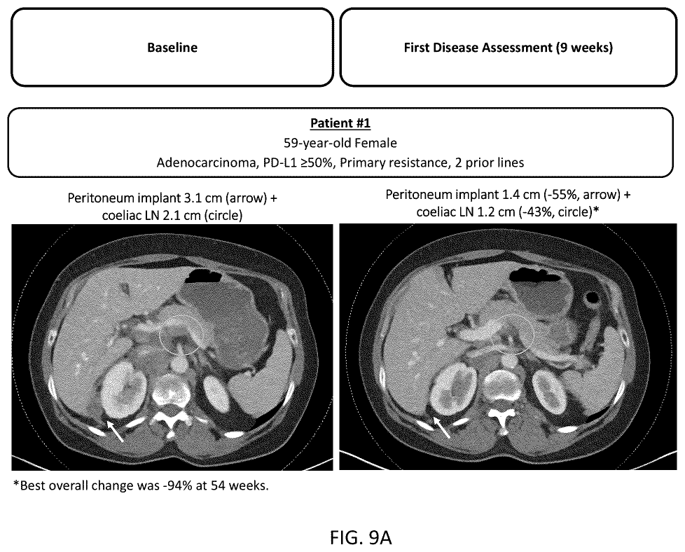

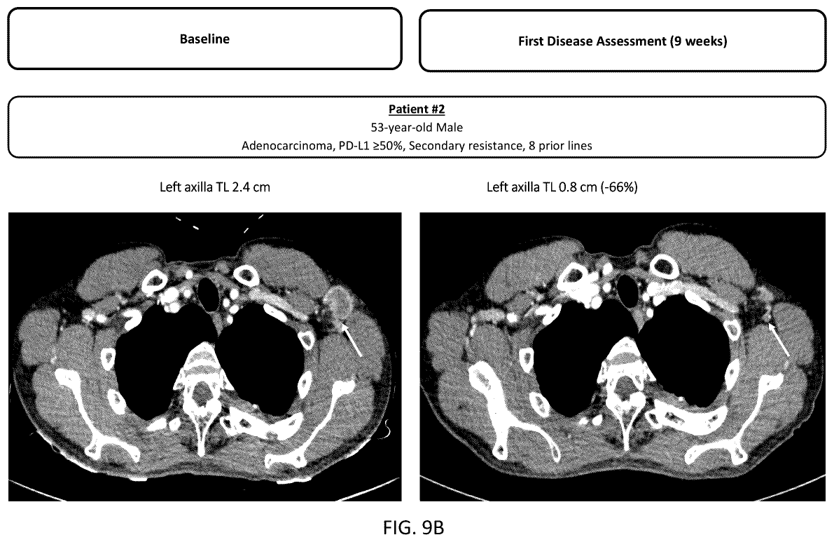

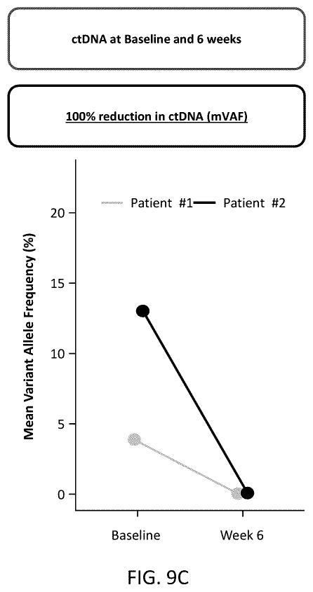

Pharmacodynamics was measured for two subjects. FIG. 9A and FIG. 9B each show a subject treated at the recommended phase 2 dose who had a confirmed radiographic response at 9 weeks. Patient 1 (FIG. 9A) is a 59-year-old female with adenocarcinoma, a PD-L1 ≥50%, with primary resistance, and 2 prior lines of treatment. Patient 2 (FIG. 9B) is a 53-year-old male with adenocarcinoma, PD-L1 ≥50%, with secondary resistance, and 8 prior lines of treatment. FIG. 9C shows that the two subjects had a corresponding 100% reduction in ctDNA at 6 weeks. The common characteristics of these patients include PR lasting ≥6 months, PD-L1 TPS ≥50%, and ECOG PS 0.

In conclusion, this study demonstrated that:
AZD2936 was safe across all doses tested. No DLTs were observed during dose escalation, and an MTD was not reached.
Antitumour activity was observed in heavily pretreated patients with CPI-resistant advanced/metastatic NSCLC treated with AZD2936, including at the recommended phase 2 dose.
The safety and preliminary efficacy observed in pretreated patients provide a rationale to further test AZD2936 in CPI-naïve patients with NSCLC.

### Example 12: Preliminary Part C Results

Nineteen subjects have been evaluated in the Part C portion of the study described in the Examples above.

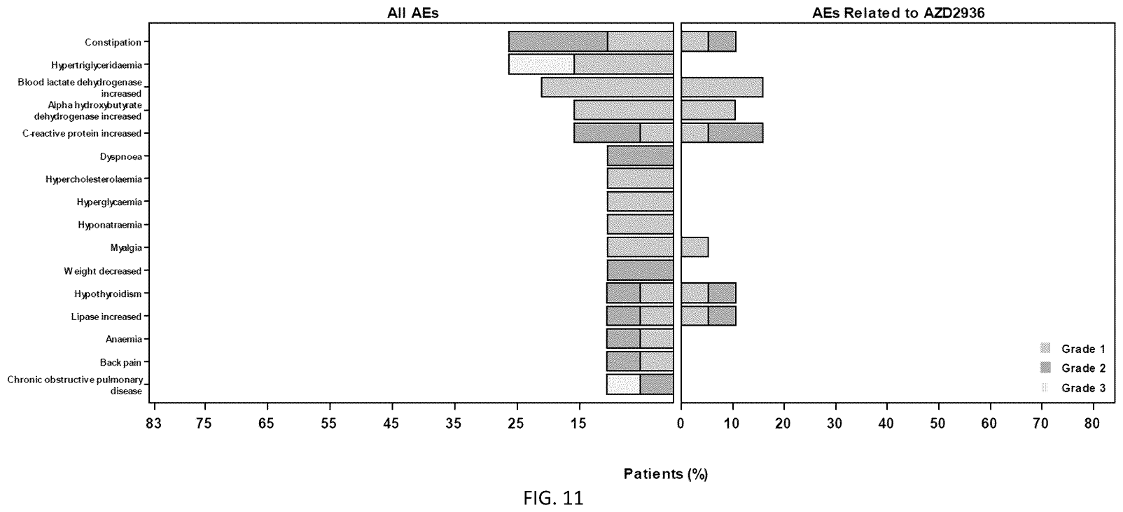

Table 11 shows the available demographics of seven subjects evaluated in Part C. Table 12 shows the available disease status for seven subjects evaluated in Part C. As shown in Table 12, most subjects present with metastatic disease and squamous histology. Adverse events occurring in greater than 10% of subjects in Part C are plotted in FIG. 11. Adverse events related to AZD2936 are shown on the right side of the plot. One SAE was observed that were possibly related to AZD2936: immune-mediated hepatitis.

TABLE 11
Demographics of Subjects Evaluated in Part C

| | Part C |
|---|---|
| | 750 mg |
| | N = 7 |
| Age, median | 65.0 |
| Sex, n ( %) | |
| Female | 1 (14.3) |
| Male | 6 (85.7) |

TABLE 12
Disease Status of Subjects Evaluated in Part C

| | Part C |
|---|---|
| | 750 mg |
| | N = 7 |
| AJCC staging at study | |
| entry, n ( %) | |
| Stage I | 0 |
| Stage IA | 0 |
| Stage IB | 0 |
| Stage IIB | 0 |
| Stage IIIA | 0 |
| Stage IIIB | 0 |
| Stage IIIC | 0 |
| Stage IV | 0 |
| Stage IVA | 6 (85.7) |
| Stage IVB | 1 (14.3) |
| Histology type, n ( %) | |
| Squamous cell | 5 (71.4) |
| carcinoma | 0 |
| Small cell carcinoma | 0 |
| Large cell carcinoma | 2 (28.6) |
| Adenocarcinoma | 0 |
| Adenosquamous | 0 |
| Other | 0 |
| Missing | |

AJCC = American Joint Committee on Cancer

Efficacy results are provided in Table 13.

TABLE 13
Initial Efficacy Results for Studies in Part C

| | Part C |
|---|---|
| Interim response- | 750 mg |
| evaluable subjects⁺ | N = 7 |
| Overall response rate, | 4 (57.1) |
| confirmed, % | |
| Complete response (CR), | 0 |
| confirmed, n ( %) | |
| Partial response (PR), | 4 (57.1) |
| confirmed, n ( %) | |
| CR or PR, unconfirmed, n | 1 (14.3) |
| ( %) | |
| Stable disease (≥8 weeks), | 3 (42.9) |
| n (%) | |
| Progression | 0 |
| Not evaluable*, n ( %) | 0 |
| Disease control rate | 100 |
| (DCR), % | |

⁺All dosed subjects who have measurable disease at baseline and who have first dose at least 9 weeks prior to data extract (where 9 weeks is the protocolled time between scans).
*No disease assessment at the time of DCO

TABLE 14
Summary of Results

| | Part C |
|---|---|
| Efficacy | N = 7 |
| Response (confirmed), n ( %) | |
| Complete response (CR) | 0 |
| Partial response (PR) | 4 (57.1) |
| Overall response rate, % | 57.1 |
| Non-response, n ( %) | |
| Stable disease | 3 (42.9) |
| Progression | 0 |
| Disease control rate (DCR), % | 100 |
| Progression-free survival, month, | — |
| median (95% CI) | |
| 6-month progression-free | — |
| survival, % | |
| Safety | N = 19 |
| Any TEAEs, n ( %) | 17 (89.5) |
| possibly related to study treatment | 11 (57.9) |
| Any TEAE ≥Grade 3, n ( %) | 4 (21.1) |
| possibly related to study treatment | 1 (5.3) |
| Any AE leading to | 1 (5.3) |
| discontinuation, n ( %) | |

As can be seen in Table 13, at 750 mg Q3W dosing, the overall response rate (ORR) was 57.1% with 3 subjects having stable disease for a DCR of 100.0%.

Tables 13 and 14 show a summary of the initial results obtained in Part C in CPI naïve subjects. Nineteen subjects with NSCLC were treated as described above with 7 that were evaluable for efficacy. The preliminary AZD2936 safety profile continues to be favorable and initial efficacy is encouraging.

### Example 13: Preliminary Part D Results

Twenty-seven subjects have been evaluated in the Part D portion of the study described in the Examples above. This includes fourteen patients in D1 (750m Q3W) and thirteen patients in D2 (1500 mg Q3W).

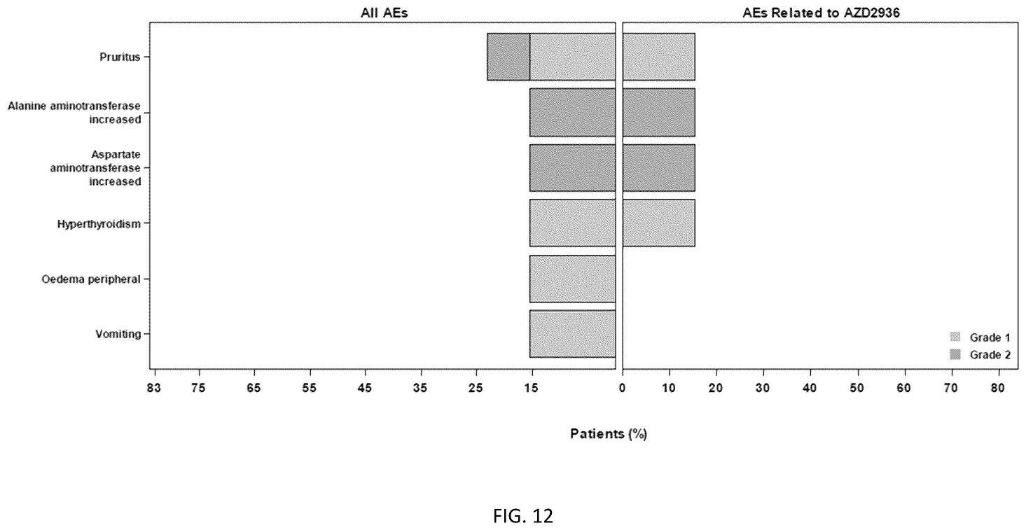

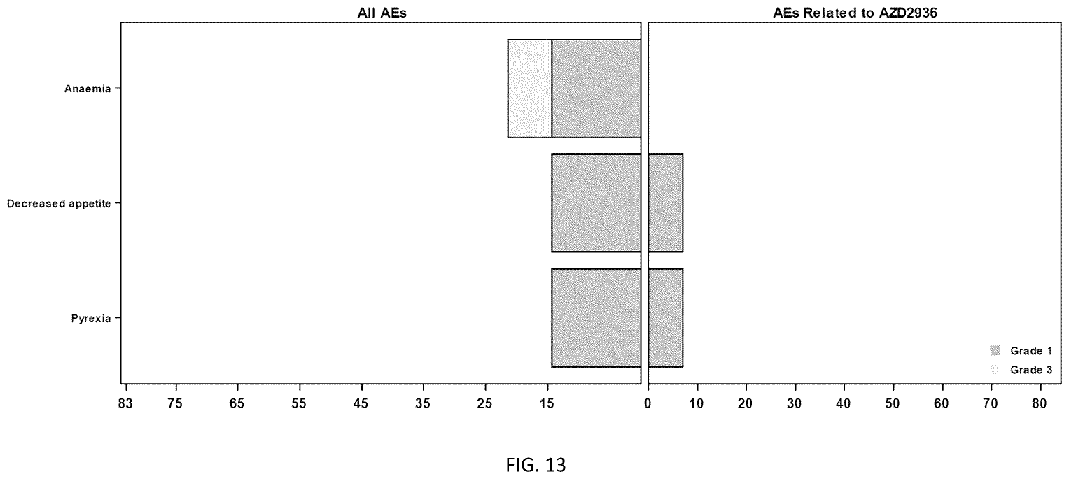

Table 15 shows the available demographics of eleven total subjects evaluated in Part D. Table 16 shows the available disease status for eleven total subjects evaluated in Part D. As shown in Table 16, most subjects present with metastatic disease and with squamous and adenocarcinoma as most common histology. Adverse events occurring in greater than 10% of subjects dosed with AZD2936 1500 mg Q3W are plotted in FIG. 12. Adverse events occurring in greater than 10% of subjects dosed with AZD2936 750 mg Q3W are plotted in FIG. 13. Adverse events related to AZD2936 are shown on the right side of both plots. One SAE was observed that were possibly related to AZD2936: alanine aminotransferase increase (occurred in Part D2 15000 mg Q3W).

TABLE 15
Demographics of Subjects Evaluated in Part D

| | Part D1 | Part D2 |
|---|---|---|
| | 750 mg | 1500 mg |
| | N = 6 | N = 5 |
| Age, median | 72.5 | 68.0 |
| Sex, n ( %) | | |
| Female | 0 | 4 (80.0) |
| Male | 6 (100) | 1 (20.0 |

TABLE 16
Disease Status of Subjects Evaluated in Part D

| | Part D1 | Part D2 |
|---|---|---|
| | 750 mg | 1500 mg |
| | N = 6 | N = 5 |
| AJCC staging at study | | |
| entry, n ( %) | | |
| Stage I | 0 | 0 |
| Stage IA | 0 | 0 |
| Stage IB | 0 | 0 |
| Stage IIB | 0 | 0 |
| Stage IIIA | 1 (16.7) | 0 |
| Stage IIIB | 0 | 0 |
| Stage IIIC | 0 | 0 |
| Stage IV | 0 | 2 (40.0) |
| Stage IVA | 2 (33.3) | 2 (40.0) |
| Stage IVB | 2 (33.3) | 1 (20.0) |
| Missing | 1 (16.7) | 0 |
| Histology type, n ( %) | | |
| Squamous cell | 1 (16.7) | 3 (60.0) |
| carcinoma | 0 | 0 |
| Small cell carcinoma | 0 | 0 |
| Large cell carcinoma | 4 (66.7) | 2 (40.0) |
| Adenocarcinoma | 0 | 0 |
| Adenosquamous | 0 | 0 |
| Other | 1 (16.7) | 0 |
| Missing | | |

AJCC = American Joint Committee on Cancer

Efficacy results are provided in Table 17.

TABLE 17
Initial Efficacy Results for Studies

| | Part D1 | Part D2 |
|---|---|---|
| Interim response- | 750 mg | 1500 mg |
| evaluable subjects⁺ | N = 7 | N = 5 |
| Overall response rate, | 2 (28.6) | 1 (20.0) |
| confirmed, % | | |
| Complete response (CR), | 0 | 0 |
| confirmed, n ( %) | | |
| Partial response (PR), | 2 (28.6) | 1 (20.0) |
| confirmed, n ( %) | | |
| CR or PR, unconfirmed, n | 1 (14.3) | 2 (40.0) |
| ( %) | | |
| Stable disease (≥8 weeks), | 5 (71.4) | 2 (40.0) |
| n (%) | | |
| Progression | 0 | 2 (40.0) |
| Not evaluable*, n ( %) | 0 | 0 |
| Disease control rate | 100 | 60.0 |
| (DCR), % | | |

⁺All dosed subjects who have measurable disease at baseline and who have first dose at least 9 weeks prior to data extract (where 9 weeks is the protocolled time between scans).
*No disease assessment at the time of DCO

TABLE 18
Summary of Results

| | Part D1 | Part D2 |
|---|---|---|
| | 750 mg Q3W | 1500 mg Q3W |
| Efficacy | N = 7 | N = 5 |
| Response (confirmed), n ( %) | | |
| Complete response (CR) | 0 | 0 |
| Partial response (PR) | 2 (28.6) | 1 (20.0) |
| Overall response rate, % | 28.6 | 20.0 |
| Non-response, n ( %) | | |
| Stable disease | 5 (71.4) | 2 (40.0) |
| Progression | 0 | 2 (40.0) |
| Disease control rate (DCR), % | 100 | 60 |
| Progression-free survival, month, | — | — |
| median (95% CI) | | |
| 6-month progression-free survival, % | — | — |
| Safety | N = 14 | N = 13 |
| Any TEAEs, n ( %) | 8 (57.1) | 10 (76.9) |
| possibly related to study treatment | 7 (50.0) | 7 (53.8) |
| Any TEAE ≥Grade 3, n ( %) | 3 (21.4) | 3 (23.1) |
| possibly related to study treatment | 0 | 0 |
| Any AE leading to discontinuation, | 0 | 0 |
| n ( %) | | |

As can be seen in Table 17, at 750 mg Q3W dosing the ORR was 28.6% with 5 subjects having stable disease for a DCR of 100.0% and at 1500 mg Q3W dosing the ORR was 20% with 2 subjects having stable disease for a DCR of 60%.

Tables 17 and 18 show a summary of the initial results obtained in Part D in CPI naïve subjects. Twenty-seven subjects with NSCLC were treated as described above with 12 that were evaluable for efficacy. The difference in ORR noted between cohorts across Part C and D are likely a reflection of sample size and will evolve over time.

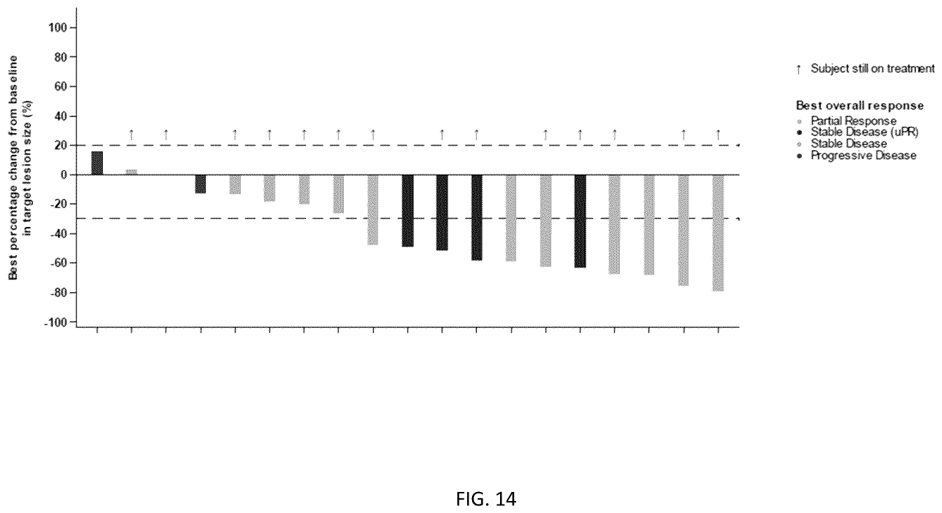

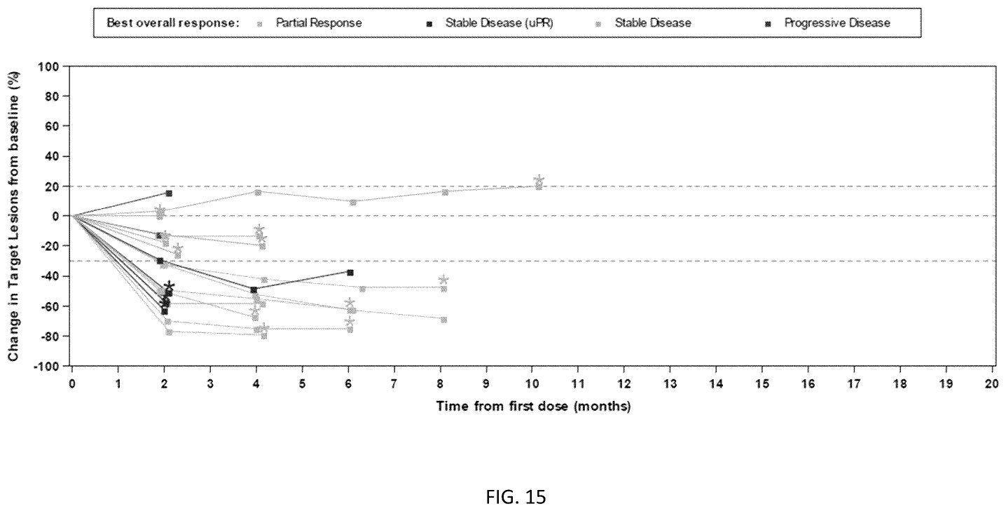

The best percentage change from baseline in target lesion in all 19 subjects with CPI naïve NSCLC dosed with AZD2936 750 mg and 1500 mg Q3W from Part C and D are presented in FIG. 14. The change of target lesion size over time in 19 subjects with CPI naïve NSCLC dosed with AZD2936 750 mg and 1500 mg Q3W from Part C and D are presented in FIG. 15. The 76preliminary AZD2936 safety profile continues to be favorable and initial efficacy is encouraging.

1. A method for treating cancer in a subject, comprising administering to the subject a bispecific binding protein that specifically binds to Programed Death-1 (PD-1) and T cell immunoreceptor with Ig and ITIM domain (TIGIT) in an amount from about 70 mg to about 1500 mg, the bispecific binding protein comprising:
a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and
b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

2-4. (canceled)

5. The method of claim 1, wherein the bispecific binding protein is administered once per treatment cycle, wherein the treatment cycle is about 7 days, about 14 days, about 21 days, about 28 days, or about 35 days; wherein the treatment cycle is repeated for up to 35 cycles.

6-14. (canceled)

15. The method of claim 1,
wherein the first binding domain of the bispecific binding protein that specifically binds to PD-1 comprises:
a. having an amino acid sequence that is at least 90% identical to the amino acid sequence of SEQ ID NO:7 and a light chain variable domain having an amino acid sequence that is at least 90% identical to the amino acid sequence of SEQ ID NO:9; and
wherein the second binding domain of the bispecific binding protein that specifically binds to TIGIT comprises:
a. a heavy chain variable domain having the amino acid sequence of SEQ ID NO:17 and a light chain variable domain having the amino acid sequence of SEQ ID NO:19.

16-22. (canceled)

23. The method of claim 1, wherein the bispecific binding protein comprises a variant Fc region.

24. The method of claim 23, wherein the variant Fc region of the bispecific binding protein comprises at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 2355, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 2545, 254T, 255L, 256E, 256F, 256M, 257C, 257M, 257N, 262I, 262A, 262T, 262E, 263I, 263A, 263T, 263M, 264L, 264I, 264W, 264T, 264R, 264F, 264M, 264Y, 264E, 265A, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 2965, 296T, 296L, 296I, 296H, 296G, 297S, 297D, 297E, 298A, 298H, 298I, 298T, 298F, 2991, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

25. The method of claim 23, wherein the variant Fc region of the bispecific binding protein comprises one or more amino acid substitutions at positions selected from 428 and 434 as numbered by the EU index as set forth in Kabat.

26. The method of claim 23, wherein the variant Fc region of the bispecific binding protein comprises one or more amino acid substitutions selected from 428L, 428F, 434A, 424F, 434W, and 434Y.

27. The method of claim 23, wherein the variant Fc region of the bispecific binding protein comprises a M252Y/S254T/T256E (YTE) mutation.

28. The method of claim 23, wherein the Fc variant region of the bispecific binding protein comprises a L234F/L235E/P331S triple mutation (TM).

29. The method of claim 23, wherein the Fc region of the bispecific binding protein is aglycosylated, deglycosylated or afucosylated.

30-31. (canceled)

32. The method of claim 23, wherein the bispecific binding protein comprises a kappa light chain constant region or a lambda light chain constant region.

33. (canceled)

34. The method of claim 23, wherein the bispecific binding protein is a humanized IgG1 antibody.

35-37. (canceled)

38. The method of claim 1, wherein the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, and lung cancer.

39. The method of claim 38, wherein the cancer is non-small cell lung cancer (NSCLC), wherein the NSCLC is advanced or metastatic.

40. (canceled)

41. The method of claim 39, wherein the subject has a PD-L1 tumor proportion score of greater than or equal to 1%.

42. (canceled)

43. The method of claim 39, wherein the subject is checkpoint inhibitor (CPI) naïve.

44. A pharmaceutical composition comprising a bispecific binding protein that specifically binds to PD-1 and TIGIT in an amount from about 70 mg to about 1500 mg, the bispecific binding protein comprising:
a) a first binding domain that specifically binds to PD-1, wherein the first binding domain comprises a heavy chain variable domain comprising a HCDR1 having the amino acid sequence of SEQ ID NO: 1, a HCDR2 having the amino acid sequence of SEQ ID NO: 2, and a HCDR3 having the amino acid sequence of SEQ ID NO: 3, and a light chain variable domain comprising a LCDR1 having the amino acid sequence of SEQ ID NO: 4, a LCDR2 having the amino acid sequence of SEQ ID NO: 5 and a LCDR3 having the amino acid sequence of SEQ ID NO: 6; and
b) a second binding domain that specifically binds to TIGIT, wherein the second binding domain comprises a heavy chain variable domain comprising a HCDR1 having the amino acid sequence of SEQ ID NO: 11, a HCDR2 having the amino acid sequence of SEQ ID NO: 12, and a HCDR3 having the amino acid sequence of SEQ ID NO: 13, and a light chain variable domain comprising a LCDR1 having the amino acid sequence of SEQ ID NO: 14, a LCDR2 having the amino acid sequence of SEQ ID NO: 15, and a LCDR3 having the amino acid sequence of SEQ ID NO: 16.

45. (canceled)

46. The pharmaceutical composition of claim 44, wherein the pharmaceutical composition comprises about 750 mg bispecific binding protein.

47. The pharmaceutical composition of claim 44, wherein the pharmaceutical composition comprises about 1500 mg bispecific binding protein.

48-55. (canceled)

56. A kit comprising the pharmaceutical composition of claim 44, further comprising instructions for administering the pharmaceutical composition.

57-64. (canceled)
